SEQUENCE LISTING (11 sequences)

SEQ ID NO: 1 - DNA, 27 bp, Artificial Sequence
  HTT genomic targeted sequence
gcgcagcgtctgggacgcaaggcgccg

SEQ ID NO: 2 - DNA, 14 bp, Artificial Sequence
  Guide polynucleotide
gacgcaaggcgccg

SEQ ID NO: 3 - DNA, 20 bp, Artificial Sequence
  Guide polynucleotide
gtctgggacgcaaggcgccg

SEQ ID NO: 4 - DNA, 22 bp, Artificial Sequence
  Guide polynucleotide
gcgtctgggacgcaaggcgccg

SEQ ID NO: 5 - DNA, 25 bp, Artificial Sequence
  Guide polynucleotide
gcagcgtctgggacgcaaggcgccg

SEQ ID NO: 6 - DNA, 51 bp, Artificial Sequence
  Single nucleotide motif
  misc_feature (26)..(26): n is c or g
gtctgcgtcagggtttccttcttttncagccccaccccgcgtgcatcccac

SEQ ID NO: 7 - DNA, 51 bp, Artificial Sequence
  Single nucleotide motif
  misc_feature (26)..(26): n is g or c
tccctcattcaggttgatgtcctaanccccagaacctcagaatgggattgt

SEQ ID NO: 8 - DNA, 51 bp, Artificial Sequence
  Single nucleotide motif
  misc_feature (26)..(26): n is c or g
tgtggcctggctaaagtaggctttantgggctcctctctgcctgcatcacc

SEQ ID NO: 9 - DNA, 51 bp, Artificial Sequence
  Single nucleotide motif
  misc_feature (26)..(26): n is g, c or t
gggcgcaggcccatgcggaaaggatnccccgccgacgcctggagcggggcg

SEQ ID NO: 10 - DNA, 51 bp, Artificial Sequence
  Single nucleotide motif
  misc_feature (26)..(26): n is c or g
attacagtctcaccacgccccgtccnctctccgttgagccccgcgccttcg

SEQ ID NO: 11 - DNA, 51 bp, Artificial Sequence
  Single nucleotide motif
  misc_feature (26)..(26): n is g or a
agcgtctgggacgcaaggcgccgtgngggctgccgggacgggtccaagatg

(19) United States
(12) Patent Application Publication
(10) Pub. No.: US 2021/0189426 A1
(43) Pub. Date: Jun. 24, 2021

(54) CRISPR INTERFERENCE BASED HTT ALLELIC SUPPRESSION AND TREATMENT OF HUNTINGTON DISEASE

(71) Applicant: THE CHILDREN'S HOSPITAL OF PHILADELPHIA, Philadelphia, PA (US)

(72) Inventors: Beverly L. DAVIDSON, Philadelphia, PA (US); Alejandro Mas MONTEYS, Philadelphia, PA (US); Shauna EBANKS, Philadelphia, PA (US)

(73) Assignee: THE CHILDREN'S HOSPITAL OF PHILADELPHIA, Philadelphia, PA (US)

(21) Appl. No.: 17/055,075

(22) PCT Filed: May 15, 2019
(86) PCT No.: PCT/US2019/032541

§ 371 Date: Nov. 12, 2020

Related U.S. Application Data
(60) Provisional application No. 62/671,969, filed on May 15, 2018.

(51) Int. Cl. C12N 15/86; C12N 15/113; C12N 9/22; A61K 45/06

(52) U.S. Cl. CPC C12N 15/86; C12N 15/113; C12N 2750/14141; A61K 45/06; C12N 9/22

(57) ABSTRACT

The invention provides expression cassettes and vectors, such as viral (e.g., AAV) vectors, comprising a first nucleic acid encoding a nuclease defective Cas 9 (dCas9) polypeptide and a second nucleic acid encoding a guide polynucleotide that targets the dCas9 polypeptide to the transcriptional start site of an allele encoding a mutant huntingtin gene (HTT)-encoded protein. Also provided are pharmaceutical composition comprising the disclosed expression cassettes and vectors, as well as methods of inhibiting expression of a mutant HTT protein and of treating Huntington's Disease and symptoms associated with the disease.

### RELATED APPLICATIONS

This application claims priority to U.S. Provisional Patent Application No. 62/671,969, filed May 15, 2018. The entire contents of the foregoing application is incorporated herein by reference, including all text, tables, sequence listing and drawings.

### INTRODUCTION

Clustered Regularly interspaced Short Palindromic Repeats (CRISPR) is a bacterial adaptive immune system that targets foreign nucleic acid sequences. Bacteria use this system to defend against infections by incorporating short fragments of the foreign DNA within the CRISPR region of its genome. The CRISPR region consists of short, repetitive palindromic spacer sequences and sequences encoding CRISPR-associated (Cas) proteins (Makarova K S, et al. Biol Direct. 2006; 1:7). When the foreign DNA fragments are expressed they behave as guide RNAs (gRNAs) to direct the Cas nuclease to the invading target, which results in cleavage of the foreign agent's DNA (Walters B J, et al. Front Genet. 2015; 6:362).

The fusion of CRISPR RNA (crRNA) and the trans-activating CRISPR RNA (tracrRNA) into a chimeric single-guide RNA (sgRNA) allows for site-specific gene editing in eukaryote genomes (FIG. 1, Cong L, et al. Science. 2013; 339(6121):819-823; Jinek M, et al. Science. 2012; 337(6096):816-821). The CRISPR type II system with the Streptococcus pyogenes Cas9 (SpCas9) nuclease is the most commonly used system, and scientists take advantage of it by designing a sgRNA targeting a specific locus, and expressing the sgRNA along with Cas9 in vitro or in vivo (Wu Y, et al. Cell Stem Cell. 2013; 13(6):659-662). Cas9 associates with the sgRNA and the complex is targeted to the desired DNA sequence (Hsu P D, et al. Cell. 2014; 157(6):1262-1278; Makarova K S, et al. Biol Direct. 2011; 6:38; Doudna J A et al. Science. 2014; 346(6213):1258096). The Cas9-sgRNA complex binds to the protospacer adjacent motif (PAM) sequence located adjacent to the sgRNA target sequence, and the two enzymatic endonuclease domains, RuvC and HNH, of Cas9 cleave both strands of DNA to generate a double-strand break (DSB) (Cong L, et al., Science. 2013; 339(6121):819-823; Hsu P D, et al. Nat Biotechnol. 2013; 31(9):827-832; Mali P, et al. Science. 2013; 339(6121):823-826). The DSB can be repaired by the two DNA repair mechanisms: non-homologous end joining (NHEJ) or homology-directed repair (HDR) (Fishman-Lobell J, et al. Mol Cell Biol. 1992; 12(3):1292-1303). NHEJ, the predominant repair pathway in mammalian cells, is error-prone and can result in insertion or deletion (indel) of nucleotides. Indels can create frameshift mutations and downstream premature stop codons leading to inactivation of the target gene (Hsu P D, et al. Cell. 2014; 157(6):1262-1278; Doudna J A, et al. Science. 2014; 346(6213):1258096). In addition to gene knockout and inactivation, targeted gene insertion is possible by CRISPR-Cas9 HDR-mediated knockin of donor DNA sequence templates, (Auer T O, et al. Methods. 2014; 69(2):142-150; Ran F A, et al. Nat Protoc. 2013; 8(11):2281-2308; Beumer K J, et al. Methods. 2014; 68(1):29-37) which can be harnessed to correct loss-of-functions mutations (Shin J W, et al. Ther Adv Neurol Disord. 2018; 11:1756285617741837). HDR may be less efficient and limited in terminally differentiated post-mitotic cells, such as neurons (Ran F A, et al. Nat Protoc. 2013; 8(11):2281-2308). However, NHEJ in neurons can occur, allowing for knock-in editing strategies. The CRISPR-Cpfl system with RNA-guided Cpfl, a nuclease like Cas9, generates DSBs with overhangs which in turn promotes NHEJ in neurons (Maresca M, et al. Genome Res. 2013; 23(3):539-546; Zetsche B, et al. Cell. 2015; 163(3):759-771; Platt R J, et al. Cell. 2014; 159(2):440-455). Finally, CRISPR-Cas9 multiplexing is possible by using multiple sgRNAs to simultaneously target different genes and pathways, or when more than one guide is required to modify a gene (Auer T O, et al. Methods. 2014; 69(2):142-150; Liu Y, et al. Insect Biochem Mol Biol. 2014; 49:35-42; Zhou J et al. Int J Biochem Cell Biol. 2014; 46:49-55; Shalem 0, et al. Science. 2014; 343(6166):84-87).

The CRISPR-Cas9 system also allows for transcriptional regulation to modulate gene expression without permanently editing the genome (FIG. 2). For this, a mutated, dead Cas9 (dCas9) lacking enzymatic endonuclease activity is used (Gilbert L A, et al. Cell. 2013; 154(2):442-451). The dCas9-sgRNA complex binds the targeted DNA sequence without creating a DSB, and is used to silence (CRISPR interference; CRISPRi) or activate (CRISPR activation; CRISPRa) gene expression Enhanced gene regulation can be achieved with effector protein domains fused to dCas9 to recruit epigenetic modifying factors (Gilbert L A, et al. Cell. 2013; 154(2):442-451; Cheng A W, et al. Cell Res. 2013; 23(10):1163-1171; Maeder M L, et al. Nat Methods. 2013; 10(10):977-979; Perez-Pinera P, et al. Nat Methods. 2013; 10(10):973-976; Tanenbaum M E, et al. Cell. 2014; 159(3):635-646). Fusing the transcriptional repressor domain Krtippel associated box (KRAB) to dCas9 is used for CRISPRi (Gilbert L A, et al. Cell. 2013; 154(2):442-451; Hu J, et al. Nucleic Acids Res. 2014; 42(7):4375-4390) and fusing the transcriptional activator domain VP64 to dCas9 is done for CRISPRa. Alternatively, the sgRNA can be modified with a MS2 coat protein (MCP) fused to an effector domain for robust transcriptional modulation (Gilbert L A, et al. Cell. 2014; 159(3):647-661; Konermann S, et al. Nature. 2015; 517(7536):583-588).

CRISPR-Cas9 delivery to cells is accomplished using standard viral and non-viral methods. Currently adeno-associated viral (AAV) vectors are the most widely used delivery method for in vivo delivery of CRISPR-Cas9. However, because AAV packaging is size-restricted to ˜4.7 kb, a dual-AAV vector system is required. One vector delivers the SpCas9 and the second vector delivers the sgRNA(s). Alternatively, Staphylococcus aureus Cas9 (SaCas9), a smaller Cas9 orthologue, can fit into a single AAV vector along with the sgRNAs (Ran F A, et al. Nature. 2015; 520(7546):186-191). Because viral delivery and constitutive expression of CRISPR-Cas9 may increase off-target effects and the risk of an immune response to the foreign bacterial protein, short-lived ribonucleoprotein (RNP) complexes of sgRNA-Cas9 are an attractive alternative for gene editing. RNP delivery is effective after direct injections into mouse cortex, striatum, and hippocampus (Staahl B T, et al. Nat Biotechnol. 2017; 35(5):431-434). Additionally, in vivo RNP delivery of an allele-specific, sgRNA-Cas9 complex was reported to correct hearing loss in a dominant deafness-associated allele in the Tmcl Beethoven mouse model (Gao X, et al. Nature. 2018; 553(7687):217-221).

The CRISPR-Cas9 system provides advantages over alternative genome editing tools, such as zinc-finger nucleases (ZFNs) and transcription activator-like effector nucleases (TALENs) (Carlson D F, et al. Mol Ther Nucleic Acids. 2012; 1:e3). Most notably, it is easily adaptable to any gene and does not require extensive optimization, although editing frequency can vary depending on the target sequence and the sgRNAs (Ansai S, et al. Biol Open. 2014; 3(5):362-371). In addition, CRISPR-Cas9 overcomes the limitations of RNA-targeting strategies for gain of function mutations, such RNA interference (RNAi) and antisense oligonucleotides (ASOs). These latter approaches, if delivered via non-viral means, require repeated treatments, and both can be prone to high off-target effects and cytotoxicity (Heidenreich M, et al. Nat Rev Neurosci. 2016; 17(1):36-44; Southwell A L, et al. Mol Ther. 2014; 22(12):2093-2106).

One drawback of the CRISPR-Cas9 system is off-target effects due to sgRNA sequence homology to other genomic sites (Wu X, et al. Quant Biol. 2014; 2(2):59-70; Wei C, et al. J Genet Genomics. 2013; 40(6):281-289). Cas9 can tolerate 3-5 mismatches depending on the distribution and number of mismatched nucleotides (Hsu P D, et al. Cell. 2014; 157(6):1262-1278; Mali P, et al. Science. 2013; 339(6121):823-826; Hu J, et al. Nucleic Acids Res. 2014; 42(7):4375-4390; Fu Y, et al. Nat Biotechnol. 2014; 32(3):279-284). Designing sgRNAs with low GC content and minimal homology to off-target sites, reducing the size of the sgRNA, and limiting the amount of Cas9 protein are all ways to minimize off-targets (Hsu P D, et al. Nat Biotechnol. 2013; 31(9):827-832; Fu Y, et al. Nat Biotechnol. 2014; 32(3):279-284; Kolli N, et al. Neurochem Int. 2018; 112:187-196). The nickase Cas9, a mutated Cas9 with only one functionally enzymatic endonuclease domain, also reduces off-targets because it cuts only one strand of the DNA. The nickase Cas9 does require two sgRNAs targeting opposite DNA strands to create a DSB for generation of the desired indel, however (Kim H, et al. Nat Rev Genet. 2014; 15(5):321-334; Ran F A, et al. Cell. 2013; 154(6):1380-1389).

Advances in engineering the CRISPR-Cas9 system have focused on optimizing editing efficiency, specificity, and Cas9 regulation. On-target specificity was improved by engineering an enhanced Cas9 (eCas9) and a high-fidelity Cas9 (SpCas9-HF1); both were reported to have reduced off-targeting (Kleinstiver B P, et al. Nature. 2016; 529(7587):490-495; Slaymaker I M, et al. Science. 2016; 351(6268):84-88). An evolved SpCas9 variant, xCas9, has improved specificity, low off-target activity, and broader PAM compatibility allowing for more broader applicability (Hu J H, et al. Nature. 2018; 556(7699):57-63). Another approach is to use a synthetic CRISPR RNA (scrRNA) to replace the wildtype crRNA, which served to reduce off-targets and enhance cleavage activity (Randar M, et al. Proc Natl Acad Sci USA. 2015; 112(51):E7110-7117). Additionally, Split-Cas9 systems have been developed to control and regulate Cas9 expression. In this scenario, Cas9 is expressed by two expression systems that can be induced to dimerize, and it has editing activity similar to wildtype SpCas9 (Truong D J, et al. Nucleic Acids Res. 2015; 43(13):6450-6458; Wright A V, et al. Proc Natl Acad Sci USA. 2015; 112(10):2984-2989; Zetsche B, et al. Nat Biotechnol. 2015; 33(2):139-14). Finally, expression regulated by small molecules, light, or temperature have been designed to control Cas9 activity (Richter F, et al. Curr Opin Biotechnol. 2017; 48:119-126).

Polyglutamine (polyQ) repeat expansion disorders such as Huntington's Disease (HD) are caused by toxic gain-of-function mutations. Huntington's disease is a neurodegenerative disease caused by an expanded CAG trinucleotide repeat in the huntingtin gene (HTT) resulting in mutant HTT protein with an expanded polyQ repeat. Huntington's disease is autosomal dominant in that a single mutant HTT allele can confer the disease.

Currently, no therapy exists for HD, and RNA interference (RNAi), a method of reducing gene expression, has emerged as a leading therapeutic option. Scientists have developed multiple strategies to co-opt the endogenous RNAi pathway for targeting specific mRNAs. Importantly, sustained co-opting may lead to cellular toxicity by interfering with the endogenous miRNA sequences. Especially for those diseases requiring chronic RNAi treatments. CRISPR-Cas9 approaches have been investigated to reduce expression of mutant HTT. Similar to what was reported in a mouse model of muscular dystrophy, CRISPR-mediated HDR has been suggested as an exon-skipping strategy to target polyQ repeats (Long C, et al. Science. 2014; 345(6201):1184-1188; Ousterout D G, et al. Nat Commun. 2015; 6:6244). Another approach is to reduce the size of the expanded CAG repeat. Using the CRISPR-Cas9 D10A nickase resulted in CAG/CTG repeat contraction in a reporter vector (Cinesi C, et al. Nat Commun. 2016; 7:13272). The problem with this approach is that other repeat-containing genes in the genome may be targeted. Replacement of the expanded polyQ repeat with a normal polyQ repeat by homologous recombination in iPSCs from HD patients improved disease phenotypes suggesting the potential for a similar HDR-mediated approach using CRISPR-Cas9 (An M C, et al. Cell Stem Cell. 2012; 11(2):253-263). However, the efficiency of an HDR-mediated approach might not be a sufficient to correct disease in humans. Furthermore, CRISPR-Cas9 targeted deletion or contraction of the HTT polyQ repeat might not restore normal protein function and it is unknown if complete knockout of both the mutant and wildtype HTT alleles will be tolerated. CRISPRi targeting the transcription start site of HTT was reported to reduce HTT mRNA and protein expression levels (Heman-Ackah S M, et al. Sci Rep. 2016; 6:28420). And CRISPR-NHEJ mediated deletion was reported to reduce mutant HTT expression in mesenchymal stem cells from the YAC128 mouse model of HD (Kolli N, et al. Int J Mol Sci. 2017; 18(4)).

### SUMMARY

Given the potency of the CRISPR/Cas9 technology for targeting both alleles, and the fact that huntingtin is an important protein for cell viability, a concern of this method is that all huntingtin will be eliminated in the cell, good and bad. Thus, an approach that selectively targets the expression of the mutant huntingtin allele would be preferred.

Disclosed herein is an epigenetic silencing approach for selective targeting of mutant huntingtin allele. The strategy takes advantage of single nucleotide polymorphisms (SNP) prevalent in the population for which a targeting sequence (PAM motif) is generated depending on the nucleotide variation. To evaluate the feasibility and tolerability of this approach, fibroblast cell lines from human HD patients have been screened for SNP heterozygosis in the Huntingtin locus. After heterozygous SNPs were identified in the mutant HTT allele, sgRNA sequences that recruit Cas9 protein and mediate epigenetic silencing to the allele-specific PAM motifs (TGG) have been designed and their efficacy determined.

In certain embodiments, the CRISPR protein is a Cas9 enzyme or variant thereof. In some embodiments, the Cas9 is derived from Streptococcus pyogenes, Staphylococcus aureus, Neisseria meningitidis, Streptococcus thermophilus, or Treponema denticola.X line.

In certain embodiments, dead Cas9 (dCas9) lacking enzymatic endonuclease activity is used to reduce or inhibit mutant HTT expression. For example, dCas9 mutations of catalytically active residues (D10 and H840) does not have nuclease activity (Zheng et al, Nat Neurosci, 21(3):44′T-454 (2018)). A dCas9-sgRNA complex binds the targeted HTT sequence without creating a break and is used to repress or inhibit expression (CRISPR interference; CRISPRi).

dCas9 can be used alone or in fusions with a transcription repressor domain to synthetically reduce or inhibit mutant HTT gene expression. Without wishing to be bound by any particular theory, dCas9 used by itself is believed to reduce or inhibit transcription through steric hindrance of the RNA polymerase or other transcriptional activators. In such an embodiment without a transcription repressor domain, CRISPRi can work independently of host cellular machinery.

In some embodiments, a dCas9 protein and a customized sgRNA designed with a complementary region to the mutant HTT allele targets dCas9 to the genomic location. In other embodiments, dCas9 is fused to a transcription repressor domain, and the fused Cas9-transcription factor can then work in concert with cellular machinery. The binding specificity is determined jointly by the complementary region on the sgRNA and a short DNA motif (protospacer adjacent motif or PAM) juxtaposed to the DNA complementary region.

Non-limiting examples of transcription repressor domains to reduce transcription include, for example, the Kruppel associated box (KRAB or SKD), the Mad mSIN3 interaction domain (SID) and the ERF repressor domain (ERD). In some such cases, the dCas9 fusion protein is targeted by the DNA-targeting guide RNA to the target mutant HTT DNA and exerts locus-specific regulation, for example, by inhibiting RNA polymerase binding to a promoter region which in turn selectively inhibits transcription activator function and/or modifying the local chromatin status, for example, when a repressor domain is used that modifies the target DNA or modifies a polypeptide associated with the target DNA.

### DESCRIPTION OF DRAWINGS

FIG. 1 shows silencing of the mutant HTT allele using SNP dependent PAMs located in the mutant HTT promoter/untranslated region. SNP=single nucleotide polymorphism; NuRD=Nucleosome Remodeling Deacetylase; HDAC=Histone Deacetylase; SET=Su(var)3-9, Enhancer-of-zeste and Trithorax; SETDB1=Domain Bifurcated Histone Lysine Methylfransferase 1; KAP1=KRAB-associated protein-1; DNMTs=DNA Methyltransferases; HP1=Heterochromatin Protein 1; HD Prom=HD Promoter; UTR=Untranslated Region.

FIG. 2 shows dCas9-mediated Gene Manipulation. Catalytically inactive dead Cas9 (dCas9) can functionally bind target DNA to impart transcriptional downregulation to suppress gene expression (top).

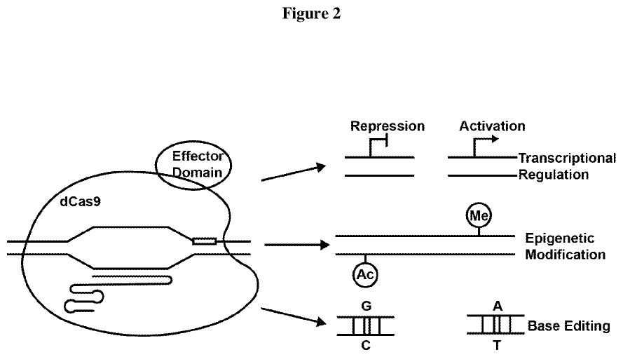

FIG. 3 shows SNP-dependent PAM motifs upstream of HTT exon1 for HTT suppression in cells homozygous for the SNP.

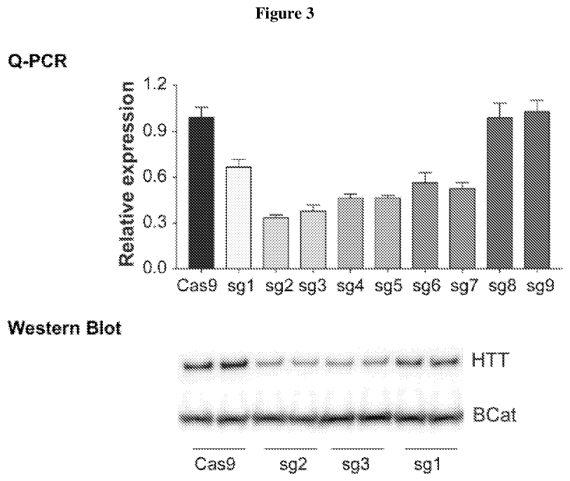

FIG. 4 shows allele suppression and discrimination of sg2 in human HD fibroblasts heterozygous for the SNP.

FIG. 5 shows exemplary AAVs for the CRISPRi approach to express dCas9 under the control of the mMECP2 promoter, and the U6/sgRNA and the CMV_EGFP_MCP_KRAB expression cassette to induce allele specific silencing of the HTT gene. mMECP2=Methyl CpG Binding Protein 2 (see, e.g., Davidson et al, Hum Mol Genet, 2018; 27(24):4303-4314); dSpCas9=mutated, nuclease-defective (“dead”) Cas9 from Streptococcus pyogenes; ITR=Inverted Terminal Repeat; pA=polyadenosine tail; sgHD; single-guide RNA targeting Huntingtin gene; hU6p=human U6 promoter (see, e.g., Romanienko et al., PLoS ONE, 2016; 11(2):1-9)); CMV=cytomegalovirus promoter; eGFP=enhanced Green Fluorescent Protein; MCP=MS2 coat protein; KRAB=Kruppel Associated Box.

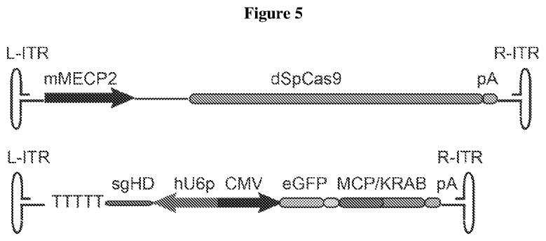

### DETAILED DESCRIPTION

Huntington's disease (HD) is a strong candidate for CRISPRi-based therapy. Huntington's disease is a progressive, ultimately fatal disorder that typically begins in adulthood. As a therapeutic strategy, efforts to lower expression of the mutant gene product prior to cell death should be highly beneficial to patients. The invention provides compositions, methods and uses of treating Huntington's disease.

In certain embodiments, a method includes administration of a subject, such as a mammal (e.g. human) with a therapeutic acid agent, e.g., a guide polynucleotide, such as a CRISPR RNA (crRNA) and a trans-activating RNA (tracrRNA), or an single guide RNA (sgRNA) alone, and a dCas9 encoded by or transcribed from an nucleic acids encoding such molecules, and optionally operably linked as an expression cassette, or a vector particle (e.g. rAAV), whereby the therapeutic agent is targeted to a Huntington's disease-associated mutant 1-ITT allele, such as a Huntington's disease-associated HTT allele comprising a single-nucleotide polymorphism.

The terms “nucleic acid” and “polynucleotide” are used interchangeably herein to refer to all forms of nucleic acid, polynucleotides, oligonucleotides, primers which are polymers of deoxyribonucleic acid (DNA) and ribonucleic acid (RNA). The term “nucleic acid” and “polynucleotide” include genomic DNA, cDNA and antisense DNA, and spliced or unspliced mRNA, rRNA tRNA, guide RNA (gRNA), single guide RNA (sgRNA), CRISPR RNA (crRNA), and trans-activating RNA (tracrRNA). Nucleic acids and polynucleotides include naturally occurring, synthetic, and intentionally altered or modified nucleic acid sequences as well as analogues and derivatives. Nucleic acids and polynucleotides can be single, double, or triplex, linear or circular, and can be of any length. In discussing nucleic acids and polynucleotides, a sequence or structure of a particular nucleic acid sequence may be described herein according to the convention of providing the sequence in the 5′ to 3′ direction.

The term “gene” is used broadly to refer to any segment of nucleic acid associated with a biological function. Thus, genes include coding sequences and/or the regulatory sequences required for their expression. For example, “gene” refers to a nucleic acid fragment that expresses mRNA, functional RNA, or specific protein. “Genes” also include nonexpressed DNA segments that, for example, form recognition sequences for other proteins. “Genes” can be obtained from a variety of sources, including cloning from a source of interest or synthesizing from known or predicted sequence information, and may include sequences designed to have desired parameters. An “allele” is one of several alternative forms of a gene occupying a given locus on a chromosome. A mutant HTT allele is an example of an alternative form of HTT.

Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues. The terms “polypeptide” and “protein” refer to a polymer of amino acids and includes full-length proteins and fragments thereof. Thus, “protein” and “polypeptide” are often used interchangeably herein. The “polypeptides” encoded by a “nucleic acid” or “polynucleotide” sequence disclosed herein, such as dCas9 polypeptides, KRAB polypeptides, Mad mSIN3 interaction domains polypeptides, ERF repressor domain polypeptides, etc., include partial or full-length native polypeptide sequences, as with naturally occurring wild-type and functional polymorphic proteins, functional subsequences (fragments) thereof, and modified forms or sequence variants thereof, so long as the polypeptide retains some degree of an activity of the natural polypeptide.

Additionally, modified forms of polypeptides, may be modified to diminish or eliminate an activity of the natural protein, and in some instances while preserving or enhancing another activity. For example, dCas9 polypeptides are modified forms of Cas9 that have been engineered to be nuclease defective while still retaining the ability to recognize and associate with a targeted locus which the dCas9 protein is targeted either through the use of a cognate crRNA and tracrRNA pair or a chimeric single guide RNA (sgRNA) to direct the dCas9 protein to the targeted locus.

A Cas9 protein that is “nuclease defective”, such as a dCas9 protein, is a variant form of a natural Cas9 protein that either has reduced endonuclease activity or is devoid of endonuclease activity relative to the endonuclease activity of the corresponding native (i.e., wild-type) Cas9 protein, Such “nuclease defective” dCas9 proteins possess either substantially reduced endonuclease activity or possess no endonuclease activity, but retain the ability to form a complex with a targeting sgRNA and to be recruited to and associate with a locus to which the dCas9-sgRNA complex is targeted in certain embodiments, a dCas9 is as disclosed in, for example, Zheng et al, Nat Neurosci, 21(3):447-454 (2018).

Such nuclease defective (dCas9) proteins may be used alone or may be used as fusions with a transcription repressor domain. Non-limiting examples of transcription repressor proteins (or transcription repressor domains from such transcription repressor proteins) to reduce transcription include, for example, the Kruppel associated box (KRAB or SKD), a Mad inSIN3 interaction domain (SID), an ERF repressor domain (ERD), a Nucleosome Remodeling Deacetylase (NuRD), a KRAB-associated protein-1 (KAN), an Su(var)3-9, Enhancer-of-zeste and Trithorax Domain Bifurcated Histone Lysine Methylfransferase 1 (SETDB 1), a DNA Methyltransferase (DNMT), a Heterochromatin Protein 1 (HP1). In certain embodiments, and without wishing to be bound by any theory, the dCas9 fusion protein is directed by a guide polynucleotide to the mutant HTT allele. The cCas9-guide RNA complex then associates with the portion of the mutant HTT allele to which it is targeted, thereby inhibiting RNA polymerase binding to the promoter region of the mutant HTT allele. This in turn inhibits the recruitment of transcription activators and chromatic relaxing proteins to the promoter region. When a repressor domain is fused to the dCas9 polypeptide, the promoter region of the targeted mutant HTT allele is actively repressed as a result of the recruitment of transcriptional repressor proteins, such, histone deacetylases, which modify chromatin such that the mutant allele is not transcribed.

The terms “modify” or “variant” and grammatical variations thereof used in such a context, mean that a nucleic acid, polypeptide or subsequence thereof deviates from a reference sequence. Modified and variant sequences may therefore have substantially the same, greater or less activity or function than a reference sequence, but at least retain partial activity or function of the reference sequence. Naturally occurring polymorphic sequences that retain at least partial function are typically not considered modified or variant since they occur in nature.

The invention therefore also includes naturally occurring and non-naturally occurring variants. Such variants include gain and loss of activity and/or function variants such as dCas9, or other nuclease defective dCas9 variants.

Non-limiting examples of modifications include one or more nucleotide or amino acid substitutions (e.g., 1-3, 3-5, 5-10, 10-15, 15-20, 20-25, 25-30, 30-40, 40-50, 50-100, or more nucleotides or residues), additions (e.g., insertions or 1-3, 3-5, 5-10, 10-15, 15-20, 20-25, 25-30, 30-40, 40-50, 50-100, or more nucleotides or residues) and deletions (e.g., subsequences or fragments) of a reference sequence. In particular embodiments, a modified or variant sequence retains at least part of a function or an activity of unmodified sequence. Such modified forms and variants can have less than, the same, or greater, but at least a part of, a function or activity of a reference sequence, for example, as described herein.

A variant can have one or more non-conservative or a conservative amino acid. sequence differences or modifications, or both. A “conservative substitution” is the replacement of one amino acid by a biologically, chemically or structurally similar residue. Biologically similar means that the substitution does not destroy a biological activity. Structurally similar means that the amino acids have side chains with similar length, such as alanine, glycine and serine, or a similar size. Chemical similarity means that the residues either have the same charge or are both hydrophilic or are both hydrophobic. Particular examples include the substitution of one hydrophobic residue, such as isoleucine, valine, leucine or methionine for another, or the substitution of one polar residue for another, such as the substitution of arginine for lysine, glutamic for aspartic acids, or glutamine for asparagine, serine for threonine, and the like. Particular examples of conservative substitutions include the substitution of a hydrophobic residue such as isoleucine, valine, leucine or methionine for another, the substitution of a polar residue for another, such as the substitution of arginine for lysine, glutamic for aspartic acids, or glutamine for asparagine, and the like. For example, conservative amino acid substitutions typically include substitutions within the following groups: glycine, alanine; valine, isoleucine, leucine; aspartic acid, glutamic acid; asparagine, glutamine; serine, threonine; lysine, arginine; and phenylalanine, tyrosine. A “conservative substitution” also includes the use of a substituted amino acid in place of an unsubstituted parent amino acid.

Such variants may be modified using recombinant DNA technology such that the nucleic acid, protein or polypeptide possesses altered or additional properties, for example, variants conferring enhanced protein stability or enhanced activity of the protein. Variants can differ from a reference sequence, such as naturally occurring nucleic acid sequences, proteins or peptides.

At the nucleotide sequence level, a variant will typically be at least about 50% identical, more typically about 70% identical, even more typically about 80% identical (90% or more identity) to the reference sequence. At the amino acid sequence level, a naturally and non-naturally occurring variant protein will typically be at least about 70% identical, more typically about 80% identical, even more typically about 90% or more identity to the reference protein, although substantial regions of non-identity are permitted in non-conserved regions (e.g., less, than 70% identical, such as less than 60%, 50% or even 40%). In other embodiments, the sequences have at least 60%, 70%, 75% or more identity (e.g., 80%, 85% 90%, 95%, 96%, 97%, 98%, 99% or more identity) to a reference sequence. Procedures for introduction of nucleotide and amino acid changes in a nucleic acid, protein or polypeptide are known to the skilled artisan (see, e.g., Sambrook et al., Molecular Cloning: A Laboratory Manual (2007)).

The term “identity,” “homology” and grammatical variations thereof, mean that two or more referenced entities are the same, when they are “aligned” sequences. Thus, by way of example, when two polypeptide sequences are identical, they have the same amino acid sequence, at least within the referenced region or portion. Where two nucleic acid sequences are identical, they have the same nucleic acid sequence, at least within the referenced region or portion. The identity can be over a defined area (region or domain) of the sequence. An “area” or “region” of identity refers to a portion of two or more referenced entities that are the same. Thus, where two protein or nucleic acid sequences are identical over one or more sequence areas or regions they share identity within that region. An “aligned” sequence refers to multiple nucleic acid or protein (amino acid) sequences, often containing corrections for missing or additional bases or amino acids (gaps) as compared to a reference sequence.

The identity can extend over the entire sequence length or a portion of the sequence. In particular aspects, the length of the sequence sharing the percent (%) identity is, e.g., 5, 6, 7, 8, 9, 10, 11, 12, 13, 14, 15, 16, 17, 18, 19, 20, etc. contiguous amino acids. In additional particular aspects, the length of the sequence sharing identity is 20 or more contiguous nucleic acids or amino acids, e.g., 20, 21, 22, 23, 24, 25, 26, 27, 28, 29, 30, 31, 32, 33, 34, 35, etc. contiguous nucleic acids or amino acids. In further particular aspects, the length of the sequence sharing identity is 35 or more contiguous nucleic acids or amino acids, e.g., 35, 36, 37, 38, 39, 40, 41, 42, 43, 44, 45, 45, 47, 48, 49, 50, etc., contiguous nucleic acids or amino acids. In yet further particular aspects, the length of the sequence sharing identity is 50 or more contiguous nucleic acids or amino acids, 50-55, 55-60, 60-65, 65-70, 70-75, 75-80, 80-85, 85-90, 90-95, 95-100, 100-110, etc. contiguous nucleic acids or amino acids.

The terms “homologous” or “homology” mean that two or more referenced entities share at least partial identity over a given region or portion. “Areas, regions or domains” of homology or identity mean that a portion of two or more referenced entities share homology or are the same. Thus, where two sequences are identical over one or more sequence regions they share identity in these regions. “Substantial homology” means that a molecule is structurally or functionally conserved such that it has or is predicted to have at least partial structure or function of one or more of the structures or functions (e.g., a biological function or activity) of the reference molecule, or relevant/corresponding region or portion of the reference molecule to which it shares homology.

The extent of identity (homology) between two sequences can be ascertained using a computer program and mathematical algorithm. Such algorithms that calculate percent (%) sequence identity (homology) generally account for sequence gaps and mismatches over the comparison region or area. For example, a BLAST (e.g., BLAST 2.0) search algorithm (see, e.g., Altschul et al., J. Mol. Biol. 215:403 (1990), publicly available through NCBI) has exemplary search parameters as follows: Mismatch −2; gap open 5; gap extension 2. For polypeptide sequence comparisons, a BLASTP algorithm is typically used in combination with a scoring matrix, such as PAM100, PAM 250, BLOSUM 62 or BLOSUM 50. Additional implementations include, but are not limited to: CLUSTAL in the PC/Gene program (available from Intelligenetics, Mountain View, Calif.); the ALIGN program (Version 2.0) and GAP, BESTFIT, PASTA (e.g., FASTA2 and FASTA3), and TFASTA in the Wisconsin Genetics Software Package, Version 8 (available from Genetics Computer Group (GCG), 575 Science Drive, Madison, Wis., USA). SSEARCH sequence comparison programs are also used to quantitate extent of identity (Pearson et al., Proc. Natl. Acad. Sci. USA 85:2444 (1988); Pearson, Methods Mol Biol. 132:185 (2000); and Smith et al., J. Mol. Biol. 147:195 (1981)). Programs for quantitating protein structural similarity using Delaunay-based topological mapping have also been developed (Bostick et al., Biochem Biophys Res Commun. 304:320 (2003)). Alignments using these programs can be performed using the default parameters.

To obtain gapped alignments for comparison purposes, Gapped. BLAST (in BLAST 2.0) can be utilized. Alternatively, PSI-BLAST (in BLAST 2.0) can be used to perform an iterated search that detects distant relationships between molecules. When utilizing BLAST, Gapped BLAST, PSI-BLAST, the default parameters of the respective programs (e.g. BLASTN for nucleotide sequences) can be used. The BLASTN program (for nucleotide sequences) uses as defaults a wordlength (W) of 11, an expectation (E) of 10, a cutoff of 100, M=5, N=−4, and a comparison of both strands. Alignment may also be performed manually by inspection.

Nucleic acids and polypeptides including modified forms can also be produced by, chemical synthesis using methods known to the skilled artisan, for example, an automated synthesis apparatus (see, e.g., Applied Biosystems, Foster City, Calif.). Peptides can be synthesized, whole or in part, using chemical methods (see, e.g., Caruthers (1980). Nucleic Acids Res, Symp. Ser. 215; Horn (1980); and Banga, A. K., Therapeutic Peptides and Proteins, Formulation, Processing and Delivery Systems (1995) Technomic Publishing Co., Lancaster, Pa.). Peptide synthesis can be performed using various solid phase techniques (see, e.g., Roberge Science 269:202 (1995); Merrifield, Methods Enzymol. 289:3(1997)) and automated synthesis may be achieved, using the ABI 431A Peptide Synthesizer (Perkin Elmer) in accordance with the manufacturer's instructions.

CRISPRi nucleic acids and polypeptides including modified forms can he made using various standard cloning, recombinant DNA technology, via cell expression or in vitro translation and chemical synthesis techniques. Purity of nucleic acid can be determined through sequencing, gel electrophoresis and the like. For example, nucleic acids can be isolated using hybridization or computer-based database screening techniques. Such techniques include, but are not limited to: (1) hybridization of genomic DNA or cDNA libraries with probes to detect homologous nucleotide sequences; (2) antibody screening to detect polypeptides having shared structural features, for example, using an expression library; (3) polymerase chain reaction (PCR) on genomic DNA or cDNA using primers capable of annealing to a nucleic acid sequence of interest; (4) computer searches of sequence databases for related sequences; and (5) differential screening of a subtracted nucleic acid library.

Expression control elements are regulatory sequences that may be present within a vector to facilitate proper heterologous nucleic acid transcription and if appropriate translation (e.g., splicing signal for introns, maintenance of the correct reading frame of the gene to permit in-frame translation of mRNA and, stop codons etc.). Typically, expression control elements are nucleic acid sequence(s), such as promoters and enhancers that influence transcription, RNA processing or stability, or translation of the associated coding sequence and therefore expression of an operably linked heterologous nucleic acid. Such elements typically act in cis but may also act in trans. Such elements, where known, are typically absent from the stuffer or filler sequence.

Expression control can be effected at the level of transcription, translation, message stability, etc. Typically, an expression control element that modulates transcription is juxtaposed near the 5′ end of the transcribed nucleic acid (i.e., “upstream”). Expression control elements can also be located at the 3′ end of the transcribed sequence (i.e., “downstream”) or within the transcript (e.g., in an intron). Expression control elements can be located at a distance away from the transcribed sequence (e.g., 100 to 500, 500 to 1000, 2000 to 5000, or more nucleotides from the nucleic acid sequence), even at considerable distances. Nevertheless, owing to the length limitations for viral vectors, such as AAV vectors, such expression control elements will typically be within 1 to 1000 nucleotides from the nucleic acid.

Functionally, expression of operably linked heterologous nucleic acid is at least in part controllable by the element (e.g., promoter) such that the element modulates transcription of the heterologous nucleic acid sequence and, as appropriate, translation of the transcript. A specific example of an expression control element is a promoter, which is usually located 5′ of the transcribed sequence. Another example of an expression control element is an enhancer, which can be located 5′, 3′ of the transcribed sequence, or within the transcribed sequence.

Expression control elements include natural and synthetic sequences as well as sequences that may be a combination of synthetic and natural sequences. The term is not exclusive to promoters.

The term “promoter” as used herein refers to a nucleotide sequence, such as a DNA sequence that is typically located adjacent to a heterologous nucleic acid sequence, usually upstream (5′) to the sequence. A promoter is operatively linked to the adjacent sequence, e.g., heterologous nucleic acid. A promoter typically increases an amount expressed from a heterologous nucleic acid as compared to an amount expressed when no promoter exists.

A “promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of the heterologous nucleic acid. This type of promoter sequence may include proximal and more distal upstream elements.

The term “enhancer” as used herein can refer to a sequence that is located adjacent to the heterologous nucleic acid. Enhancer elements are typically located upstream of a promoter element but also function and can be located downstream of or within a DNA sequence a heterologous nucleic acid). Hence, an enhancer element can be located 100 base pairs, 200 base pairs, or 300 or more base pairs upstream or downstream of a heterologous nucleic acid. An enhancer may also be an innate element of a promoter, Enhancers are often capable of operating in both orientations, either upstream or downstream from the promoter. Enhancers typically increase expression of a heterologous nucleic acid above increased expression afforded by a promoter element.

Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions.

Expression control elements (e.g., promoters) include those active in a particular tissue or cell type, referred to herein as a “tissue-specific expression control elements/promoters.” Tissue-specific expression control elements are typically active in specific cell or tissue (e.g., active in brain, central nervous system, spinal cord, liver, eye, retina, hone, muscle, lung, pancreas, heart, kidney cell, etc.). Expression control elements are typically active in these cells, tissues or organs because they are recognized by transcriptional activator proteins, or other regulators of transcription, that are unique to a specific cell, tissue or organ type. Examples of CNS-specific promoters include those isolated from the genes myelin basic protein (MBP), glial fibrillary acid protein (GFAP), and neuron specific enolase (NSE).

In certain embodiments, a CNS- or brain-specific (e.g., a brain tissue- or brain bell-specific) promoter is used in accordance with the invention. Suitable brain-specific promoters include a aldolase C promoter, a tyrosine hydroxylase promoter, a human or mouse Cluster of Differentiation 90 (Thy1) gene promoter, a human or mouse Neuron specific enolase (NSF) promoter, a human or mouse Rhombotin 1 promoter, a human or mouse Neurofilament Low (NF-L) promoter; a human or mouse dopamine beta-hydroxylase (DBH) promoter; a human or mouse Synapsin-1 promoter, a 67 kDa glutamic acid decarboxylase (GAD67) promoter, a homeobox. Dlx5/6 promoter, a glutamate receptor 1 (GluR1) promoter, a preprotachykinin 1 (Tac1) promoter, and a dopaminergic receptor 1 (Drd1a) promoter (see, e.g., WO 2002/049714; WO 2013/139676; and Delzor et al., Hum Gene Ther Methods, 23(4):242-254 (2012)).

Expression control elements also include ubiquitous or promiscuous promoters/enhancers which are capable of driving expression of a nucleic acid sequence in many different cell types. Such elements include, but are not limited to the cytomegalovirus (CMV) immediate early promoter/enhancer sequences, the Rous sarcoma virus (RSV) promoter/enhancer sequences and the other viral promoters/enhancers active in a variety of mammalian cell types, or synthetic elements that are not present in nature (see, e.g., Boshart et al, Cell, 41:5:21-530 (1985)), the SV40 promoter, the dihydrofolate reductase promoter, the cytoplasmic β-actin promoter and the phosphoglycerol kinase (PGK) promoter. Further non-limiting examples of promoters useful in the invention include mouse U6 RNA promoters, human U6 RNA promoters, synthetic human H1RNA promoters, RNA polymerase II and RNA polymerase III promoters.

Expression control elements also can confer expression in a manner that is regulatable, that is, a signal or stimuli increases or decreases expression of the operably linked heterologous nucleic acid. A regulatable element that increases expression of the heterologous nucleic acid in response to a signal or stimuli is also referred to as an “inducible element” (i.e., is induced by a signal).

Expression control elements also include native elements(s) for the heterologous nucleic acid. A native control element (e.g., promoter) may be used when it is desired that expression of the heterologous nucleic acid should mimic the native expression. The native element may be used when expression of the heterologous nucleic acid is to be regulated temporally or developmentally, or in a tissue-specific manner, or in response to specific transcriptional stimuli. Other native expression control elements, such as introns, polyadenylation sites or Kozak consensus sequences may also be used.

The term “operable linkage” or “operably linked” refers to a physical or functional juxtaposition of the components so described as to permit them to function in their intended manner. In the example of an expression control element in operable linkage with a heterologous nucleic acid, the relationship is such that the control element modulates expression of the heterologous nucleic acid. More specifically, for example, two DNA sequences operably linked means that the two DNAs are arranged (cis or trans) in such a relationship that at least one of the DNA sequences is able to exert a physiological effect upon the other sequence.

“Expression cassette” as used herein means a nucleic acid sequence that is itself comprised of one or more nucleic acid sequences encoding and directing the expression of one or more polynucleotides and/or one or more polypeptides in an appropriate host cell. Expression cassettes may also include one or more promoters and other expression control sequences operably linked to the one or more nucleic acid sequences of interest, and may also be operably linked to termination signals. The coding region of one of the nucleic acids of interest may encode a functional polynucleotide of interest, for example a guide polynucleotide, such as a crRNA, a tracrRNA, or an sgRNA. The coding region of another the nucleic acid of interest may encode a functional polypeptide of interest, for example a dCasp polypeptide.

The expression cassette including a nucleic acid sequence may be chimeric. The expression cassette may also be one that is naturally occurring but has been obtained in a recombinant form useful for heterologous expression. The expression of the sequence in the expression cassette may be under the control of a constitutive promoter or of a regulatable promoter that initiates transcription only when the host cell is exposed to some particular stimulus. In the case of a multicellular organism, the promoter can also be specific to a particular tissue or organ, such as CNS (e.g. brain) or may be specific to a particular stage of development.

Such expression cassettes can include a transcriptional initiation region linked to a nucleotide sequence of interest. Such an expression cassette is provided with a plurality of restriction sites for insertion of the gene of interest to be under the transcriptional regulation of the regulatory regions. The expression cassette may additionally contain selectable marker genes.

“Expression” refers to the transcription and/or translation of a heterologous nucleic acid, polynucleotide, a transgene, a polymorphic allele, an allele comprising a single nucleotide polymorphism, or an endogenous gene in cells. For example, in the case of an endogenous gene expression may refer to the expression of the target endogenous polymorphic allele sought to be silenced.

The term “vector” includes, inter alia, a plasmid, virus (e.g., AAV vector), cosmid, or other vehicle in double or single stranded linear or circular form that can be manipulated by insertion or incorporation of a nucleic acid. A “vector” can introduce/transfer nucleic acid sequences into a prokaryotic or eukaryotic host, such as cells of a mammal, either by integration into the cellular genome or extrachromosomally (e.g., autonomous replicating plasmid with an origin of replication). Vectors can be used to transcribe or translate the introduced/transferred nucleic acid in cells. Vectors can also be used for genetic manipulation (i.e., “cloning vectors”). A vector or plasmid generally contains at least an origin of replication for propagation in a cell and optionally additional elements. Optional elements include but are not limited to a heterologous nucleic acid sequence, expression control element (e.g., a promoter, enhancer), selectable marker (e.g., antibiotic resistance), transcription termination signals (poly-adenylation sequence), translation stop signals (stop codons).

As used herein, the term “recombinant,” as a modifier of a vector such as a viral (e.g., AAV) vector, as well as a modifier of sequences such as “recombinant” nucleic acid sequences and polypeptides, means that the compositions have been manipulated (i.e., engineered) in a fashion that generally does not occur in nature. A nucleic acid sequence example of a recombinant vector, such as AAV vector would be where a nucleic acid sequence that is not normally present in the wild-type viral (e.g., AAV) genome is within the viral (e.g., AAV) particle and/or viral (e.g., AAV) genome. Although the term “recombinant” is not always used herein in reference to vectors such as viral (e.g., AAV) vectors, as well as sequences such as nucleic acid sequences and polypeptides, hybrids and chimeras, recombinant forms of vectors (e.g., AAV), and sequences including nucleic acids, nucleic acid sequences and polypeptides, hybrids and chimeras, are expressly included in spite of any such omission.

In particular embodiments, a recombinant vector (e.g., AAV) is a parvovirus vector. Parvoviruses are small viruses with a single-stranded DNA genome. “Adeno-associated viruses” (AAV) are in the parvovirus family.

Parvoviruses including AAV are viruses useful as gene therapy vectors as they can penetrate cells and introduce nucleic acid/genetic material. AAV can penetrate cells and introduce nucleic acid/genetic material so that the nucleic acid/genetic material is stably maintained in cells. Because AAV are not associated with pathogenic disease in humans, AAV vectors are able to deliver invention CRISPRi guide RNA and dCas9 nucleic acid sequences to human patients without causing substantial AAV pathogenesis or disease.

An “AAV virus” or “AAV viral particle” refers to a viral particle composed of at least one AAV capsid protein (or optionally all of the three AAV capsid proteins, VP1, VP2 and VP3) and an encapsidated nucleic acid. If the particle encapsidates a heterologous nucleic acid (i.e., a non-native sequence other than a wild-type AAV genome such as a transgene to be delivered to a cell), it is typically referred to as “rAAV.” Incorporation of a heterologous nucleic acid sequence therefore defines the viral vector (e.g. AAV) as a “recombinant” vector, which in the case of AAV the particle can be referred to as a “rAAV.” Or as an “rAAV vector.”

A recombinant viral vector, such as “AAV vector” is derived from the wild type genome of a virus, such as AAV by using molecular methods to remove the wild type genome from the virus (e.g., AAV), and replacing with a non-native nucleic acid, such as a heterologous nucleic acid sequence (e.g., a therapeutic gene). Typically, for AAV one or both inverted terminal repeat (ITR) sequences of the wild type AAV genome are retained in the AAV vector. A viral vector (e.g., AAV) is distinguished from a viral (e.g., AAV) genome, since all or a part of the viral genome has been replaced with a heterologous nucleic acid sequence, which heterologous sequence is typically a non-native nucleic acid with respect to the viral (e.g., AAV) genomic nucleic acid.

For a recombinant vector, a vector genome refers to the portion of the vector plasmid that is packaged or encapsidated by virus (e.g., AAV), which contains the heterologous nucleic acid sequence. The plasmid portion of the recombinant vector includes the backbone used for helper cell transfection and cell production of virus that packages/encapsidates the vector genome, but is not itself packaged or encapsidated by virus (e.g., AAV). In addition, such vector genomes can be included (packaged) within a virus, such as an adeno-associated virus (e.g., AAV), which is also referred to herein as a “particle” or “virion” for subsequent infection (transformation) of a cell, ex vivo, in vitro or in vivo.

An “AAV ITR” is a region found at each end of the AAV genome which functions together in cis as origins of DNA replication and as packaging signals for the virus. AAV ITRs, together with AAV rep coding region, provide for the efficient excision and rescue from, and integration of a nucleic acid sequence positioned between two flanking ITRs into a. mammalian cell genome.

To produce rAAV virions, an AAV expression vector is introduced into a suitable host cell using known techniques, such as by transfection. A number of transfection techniques are can be used. See, e.g., Sambrook et al. (1989) Molecular Cloning, a laboratory manual, Cold Spring Harbor Laboratories, New York. Suitable transfection methods include calcium phosphate co-precipitation, direct micro-injection into cultured cells, electroporation, liposome mediated gene transfer, lipid-mediated transduction, and nucleic acid delivery using high-velocity microprojectiles.

Recombinant vectors include vectors (e.g., rAAV) may include a filler or stuffer sequence having a size at, approaching or slightly greater in length than the natural packaging capacity of the virus (AAV), and methods of using such recombinant vectors (e.g., rAAV), for example, to produce recombinant virus particles having reduced or eliminated residual DNA impurities. Recombinant vectors as set forth herein may also include an additional filler or stuffer nucleic acid sequence that resizes or adjusts the length to near or at the normal size of the virus genomic sequence that is packaged or encapsidated to form infectious virus particles as disclosed, for example, in Patent Cooperation Treaty publication number WO 2014/144486. In various embodiments, a filler or stuffer nucleic acid sequence is an untranslated (non-protein encoding) segment of nucleic acid.

As disclosed herein, AAV vectors typically accept inserts of DNA having a defined size range which is generally about 4 kb to about 5.2 kb, or slightly more. When there are shorter heterologous sequences in the vector, inclusion of a stiffer or filler in the insert fragment (e.g., vector genome) will provide a length acceptable for AAV vector packaging. Thus, in accordance with the invention vectors in which there is included a stuffer or filler in the packaged (encapsidated) portion (vector genome) to provide a size approaching the natural packaging capacity of the virus AAV) are provided.

In particular embodiments of an AAV vector, a vector sequence has a length less than 4.7 Kb and the filler or smiler sequence has a length that when combined (e.g., inserted into a vector) with the vector sequence has a total length ranging from about 3.0-5.5 Kb, or ranging from about 4.0-5.0 Kb, or ranging from about 4.3-4.8 Kb. For example, length of a vector for AAV particle packaging can be up to about 5.2 kb.

In various embodiments, in the context of an AAV vector a filler or staffer nucleic acid sequence has a sequence length in a range of 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, or 5,500-6,000 contiguous nucleotides in length.

Vectors (e.g., AAV), and particles (e.g., AAV) including such vector genomes, include any virus strain or serotype, and subgroups and variants thereof. As used herein, the term “serotype” is a distinction used to refer to a virus (e.g., VAV) having a capsid that is serologically distinct from other virus (e.g., AAV) serotypes.

A “serotype” is traditionally defined on the basis of a lack of cross-reactivity between antibodies to one virus as compared to another virus. Such cross-reactivity differences are usually due to differences in capsid protein sequences/antigenic determinants (e.g., due to VP2, and/or VP3 sequence differences of AAV serotypes). Under the traditional definition, a serotype means that the virus of interest has been tested against serum specific for all existing and characterized serotypes for neutralizing activity and no antibodies have been found that neutralize the virus of interest. As more naturally occurring virus isolates of are discovered and/or capsid mutants generated, there may or may not be serological differences with any of the currently existing serotypes. Thus, in cases where the new virus (e.g., AAV) has no serological difference, this new virus (e.g., AAV) would be a subgroup or variant of the corresponding serotype. In many cases, serology testing for neutralizing activity has yet to be performed on mutant viruses with capsid sequence modifications to determine if they are of another serotype according to the traditional definition of serotype. Accordingly, for the sake of convenience and to avoid repetition, the term “serotype” broadly refers to both serologically distinct viruses (e.g., AAV) as well as viruses (e.g., AAV) that are not serologically distinct that may be within a subgroup or a variant of a given serotype.

By way of a non-limiting example, AAV include various naturally and non-naturally occurring serotypes. Such non-limiting serotypes include, for example, AAV1, AAV2, AAV3, AAV3B, AAV4, AAV5, AAV6, AAV7, AAV8, AAV9, AAV10, AAV11, Rh10, Rh74, and AAV-2i8. Again, for the sake of convenience serotypes include AAV with capsid sequence modifications that have not been fully characterized as being a distinct serotype, and may in fact actually constitute a subgroup or variant of a known serotype.

Accordingly, invention recombinant vector (e.g., AAV), and particles that include packaged or encapsidated vector genomes, as well as methods and uses thereof, include any viral strain or serotype. As a non-limiting example, a recombinant vector (e.g., AAV) can be based upon any AAV genome, such as AAV1, AAV2, AAV3, AAV3B, AAV4, AAV5, AAV6, AAV7, AAV8, AAV9, AAV10, AAV11, Rh10, or Rh74, for example. A particle (virus) that packages (also referred to as encapsidates) a recombinant vector (e.g., AAV) genome can be based upon any AAV serotype such AAV1, AAV2, AAV3, AAV3B, AAV4, AAV5, AAV6, AAV7, AAV8, AAV9, AAV10, AAV11, Rh10, or Rh74, for example.

Such vectors and particles can be based on the same strain or serotype (or subgroup or variant), or be different from each other. As a non-limiting example, a recombinant vector (e.g., AAV) plasmid based upon AAV2 serotype genome (e.g., AAV2 ITRs) can be identical to one or more of the capsid proteins that package the vector, in which case at least one of the three VP1, VP2 and VP3 capsid proteins would also be AAV2. In addition, a recombinant vector (e.g., AAV) plasmid based upon AAV2 serotype genome (e.g., AAV2 ITRs) can be a distinct serotype from one or more of the capsid proteins that package the vector, in which case at least one of the three capsid proteins could be a non-AAV2 capsid, such as AAV1, AAV3, AAV3B, AAV4, AAV5, AAV6, AAV7, AAV8, AAV9, AAV10, AAV11, Rh10, Rh74, or AAV-2i8 capsid, for example.

An AAV serotype may be selected/designed according to a desired route of administration, for example, and without limitation, for systemic administration, an AAV vector capable of crossing the blood-brain barrier may be used (e.g., AAV9, or a chimeric AAV vector having AAV9 capsid proteins). The invention also includes compositions, methods and uses in which AAV vector is administered to the bloodstream using serotypes capable of traversing the blood-brain barrier.

In certain embodiments. AAV vector is administered directly to the CNS or to the brain using serotypes capable of transducing CNS or brain cells. In certain embodiments, an AAV2 capsid is employed in accordance with the invention.

In certain embodiments, recombinant vector (e.g., AAV), and particles with the packaged (encapsidated) portion (vector genome) include hybrids or chimeras. As a non-limiting example, a hybrid vector genome can be a mixed serotype, e.g., one virus genome serotype, such as an AAV2 serotype and a non-AAV2 serotype, for example, an AAV2 flanking (5′ or 3′) ITR, and a non-AAV2 flanking (5′ or 3′) ITR. More particularly, as an example, a vector genome that is hybrid AAV serotype, could be an AAV2 flanking (5′ or 3′) ITR and an AAV1, AAV3, AAV3B, AAV4, AAV5, AAV6, AAV7, AAV8, AAV9, AAV10, AAV11, Rh10, or Rh74 (5′ or 3′) ITR. As another non-limiLing example, a vector can be a hybrid AAV serotype, such as an AAV2 capsid and a non-AAV2 capsid, for example, an AAV2 VP1, VP2 or VP3, and a non-AAV2 VP1, VP2 or VP3. More particularly, a hybrid or chimeric vector genome or virus that is an AAV serotype, could be an AAV2 VP1, VP2 or VP3 and a AAV1, AAV2, AAV3, AAV3B, AAV4, AAV5, AAV6, AAV7, AAV8, AAV9, AAV10, AAV11, Rh10, Rh74, or AAV-2i8 VP, VP2 or VP3.

Recombinant vectors (e.g., AAV) and particles (e.g., that include AAV capsid proteins) as set forth herein include those having nucleic acid sequence, polypeptide or subsequence thereof that has less than 100% sequence identity to a reference sequence. In various embodiments, a sequence that has less than 100% sequence identity to a reference sequence is at least 70% or more (e.g., 70-75%, 75-80%, 80-85%, 85-90%, 90-95%, 96%, 97%, 98%, 99%, 99.5%, etc.) identical to a reference sequence, for example, 80% or more (e.g., 80-85%, 85-90%, 90-95%, 96%, 97%, 98%, 99%, 99.5%, etc.) identical to a reference sequence. For example, reference sequences also include dCas9 sequences and HTT mutant allele guide sequences as set forth herein.

Reference sequences include heterologous nucleic acid sequences, vector sequences, expression control elements, the additional elements that can be included or combined with a vector as set forth herein. Reference sequences include any of AAV1, AAV2, AAV3, AAV3B, AAV4, AAV5, AAV6, AAV7, AAV8, AAV9, AAV10, AAV11, Rh10, Rh74, or AAV-2i8 VP1, VP2, and/or VP3 capsid sequence, or 5′ or 3′ ITR of AAV1, AAV2, AAV3, AAV3B, AAV4, AAV5, AAV6, AAV7, AAV8, AAV9, AAV10, AAV11, Rh10, or Rh74. Such capsid sequences and 5′ and 3′ ITR for AAV1, AAV2, AAV3, AAV3B, AAV4, AAV5, AAV6, AAV7, AAV8, AAV9, AAV10, AAV11, Rh10, Rh74 and AAV-2i8 are known in the art.

Recombinant vector (e.g., AAV), including AAV1, AAV2, AAV3, AAV3B, AAV4, AAV5, AAV6, AAV7, AAV8, AAV9, AAV10, AAV11, Rh10, Rh74 or AAV-2i8 and related hybrid and chimeric sequences, can be constructed using recombinant techniques that are known to the skilled artisan, to include a heterologous nucleic acid sequence transgene flanked with one or more functional AAV ITR sequences.

Such vectors can have one or more of the wild-type AAV genes deleted in whole or in part, for example, a rep and/or cap gene, but retain at least one functional flanking ITR sequence, as necessary for the rescue, replication, and packaging of the AAV vector genome by capsid proteins. Thus, an AAV vector genome includes sequences required in cis for replication and packaging (e.g., functional ITR sequences).

Suitable host cells for packaging rAAV virions include microorganisms, yeast cells, insect cells, and mammalian cells, that can be, or have been, used as recipients of a heterologous nucleic acid. Host cells includes progeny of the original cell which was transfected. Particular examples of suitable cells are stable human cell lines, such as 293 (American Type Culture Collection under Accession Number ATCC CRL1573). The human cell line 293 is a human embryonic kidney cell line that has been transformed with adenovirus type-5 DNA fragments, and expresses the adenoviral E1a and E1b genes. The 293 cell line is readily transfected, and provides a convenient platform in which to produce rAAV virions.

An “AAV rep coding region” is the region of the AAV genome which encodes the replication proteins Rep 78, Rep 68, Rep 52 and Rep 40. These Rep proteins have been shown to possess many functions, including recognition, binding and nicking of the AAV origin of DNA replication, DNA helicase activity and modulation of transcription from AAV (or other heterologous) promoters. The Rep proteins are collectively required for replicating the AAV genome. Homologs of the AAV rep coding region include human herpesvirus 6 (HHV-6) rep gene.

The “AAV cap coding region” is the region of the AAV genome which encodes the capsid proteins VP1, VP2, and VP3. These capsid proteins supply the packaging functions that are collectively required for packaging the viral genome.

AAV helper functions can be introduced into the host cell by transfecting the host cell with an AAV helper construct either prior to, or concurrently with, transfection of the AAV vector. AAV helper constructs thus provide at least transient expression of AAV rep and/or cap genes to complement missing AAV functions that are necessary for productive AAV infection. AAV helper constructs lack AAV ITRs and can neither replicate nor package themselves. A number of AAV helper constructs have been described, such as plasmids pAAV/Ad and pIM29+45 which encode both Rep and Cap expression products. A number of other vectors have been described which encode Rep and/or Cap expression products.

Recombinant vectors (e.g., AAV) in which the packaged (encapsidated) portion (referred to as the “vector genome” or simply “vector”) has a size approaching the natural packaging capacity of the virus (e.g., AAV) can be used to transfer/deliver heterologous nucleic acid sequences, such as coding sequences (genes) for proteins that provide a desirable or therapeutic benefit, as well as nucleic acids that reduce or inhibit expression of an undesirable or defective (e.g., pathologic) gene, thereby treating a variety of diseases. For example, a recombinant vector (e.g., AAV) (e.g., mutant HTT) can be used to transfer/deliver therapeutic genes to treat a genetic deficiency disease, such as diseases of the central nervous system including neurodegenerative diseases, such as spinocerebellar ataxia and Huntington's disease and for other therapeutic purposes.

The term “consists essentially of” or “consisting essentially of” when referring to a particular nucleic acid sequence or amino acid sequence means a sequence having the properties of a given sequence, e.g., a stuffer or filler as set forth herein. For example, when used in reference to an nucleic acid sequence, the phrase includes the sequence per se and molecular modifications that would not affect the basic and novel characteristics of the sequence.

The polynucleotide sequences, polypeptide sequences, promoters, enhancers, expression control sequences, and expression cassettes, alone or in combination with guide RNA encoding and dCas9 encoding encoding nucleic acid sequences, expression control elements and additional elements, and vectors, including recombinant AAV vectors, can be isolated or substantially purified. In the context of the invention, an “isolated” or “purified” molecule is made by the hand of man or exists apart from its native environment and is therefore not a product of nature. Generally, isolated molecules may exist in a purified form or in a non-native environment such as, for example, a transgenic host cell. For example, an “isolated” or “purified” polynucleotide sequence or polypeptide sequence is substantially free of other cellular material, or culture medium when produced by recombinant techniques, or substantially free of chemical precursors or other chemicals when chemically synthesized, or substantially free of one or more materials with which they normally associate with in nature, for example, one or more protein, nucleic acid, lipid, carbohydrate, cell membrane.

The term “isolated” does not exclude combinations produced by the hand of man, for example, a recombinant vector (e.g., rAAV), or virus particle that packages invention guide polynucleotides and dCas9 polynucleotides or an invention expression cassette comprising guide polynucleotides and/or dCas9 polynucleotides. The term “isolated” also does not exclude alternative physical forms of the composition, such as hybrids/chimeras, multimers/oligomers, modifications (e.g., phosphorylation, glycosylation, lipidation) or derivatized forms, or forms expressed in host cells produced by the hand of man.

As set forth herein, recombinant vector (e.g., AAV) can be used to deliver exogenous nucleic acid sequences (e.g., heterologous nucleic acid sequences) to cells ex vivo, in vitro and in vivo. Such heterologous nucleic acid sequences can encode proteins such that the cells into which the nucleic acid is delivered express the encoded proteins. For example, a recombinant vector (e.g., AAV) can include a heterologous nucleic acid sequence encoding a desired (e.g., therapeutic) CRISPRi guide RNA and/or dCas polypeptide.

Invention recombinant vectors (e.g., AAV) can be used to introduce/deliver invention CRISPRi polynucleotides stably or transiently into cells and progeny thereof. As set forth herein, a “transgene” refers to a heterologous nucleic acid sequence that is suitable for introduction into a cell or organism.

For example, in a cell having a transgene, the transgene has been introduced/transferred by way of vector (e.g., AAV) “transduced” into the cell. The terms “transduce” and “transfect” refer to introduction of a molecule such as a tiller or stuffer sequence alone or in combination with other elements, such as a heterologous nucleic acid, vector, etc., into a cell or host organism. The term “transduction” is generally used to refer to infecting cells with viral particles. The term “transfection” is generally used to refer to the delivery of DNA into eukaryotic (e.g., mammalian) cells. Accordingly, a “transduced” or “transfected” cell (e.g., in a mammal, or a cell or tissue or organ), means a genetic change in a cell following incorporation of an exogenous molecule, for example, a heterologous nucleic acid sequence (e.g., a transgene) or protein into the cell. A “transduced” or “transfected” cell can be a cell into which, or a progeny thereof, in which an exogenous molecule has been introduced, for example. The cell(s) can be propagated and the introduced protein expressed, or heterologous nucleic acid transcribed.

For gene therapy methods and uses, a transduced or transfected cell can be in a subject such as a mammal. Typically, introduction into cells in a subject is by way of a vector, such as AAV.

The invention provides methods of delivering a heterologous nucleic acid to a cell. In one embodiment, a method includes administering to the cell an AAV particle containing a vector comprising a heterologous nucleic acid inserted between a pair of AAV inverted terminal repeats, thereby delivering the nucleic acid to the cell. Administration to the cell can be accomplished by any means. The particle can be allowed to remain in contact with the cells for any desired length of time. For in vitro methods, the AAV vector can be administered to the cell by standard viral transduction methods. For example, cells can be transduced in vitro by combining recombinant AAV vector with CNS cells e.g., in appropriate media, and screening for those cells harboring the nucleic acid of interest using conventional techniques. Transduced cells can then be formulated into pharmaceutical compositions, described more fully below, and the composition introduced into the subject by various techniques.

Titers of AAV vector to administer can vary, particularly depending upon the cell type, but will be typical of that used for AAV transduction in general. The cells can include any desired cell in humans as well as other large (e.g., non-rodent) mammals, such as primates, horse, sheep, goat, pig, and dog.

Transduced and transfected cells may be produced using a variety of methods. Biological methods include the use of DNA (e.g., AAV) and RNA viral vectors. For mammalian gene therapy, as described herein, it is desirable to use an efficient means of inserting a transgene into the host genome. Viral vectors, and especially AAV and lentiviral vectors, have become the most widely used method for inserting genes into mammalian, e.g., human cells.

To confirm the presence of the transgene in the host cell, a variety of assays may be performed. Such assays include, for example, “molecular biological” assays such as Southern and Northern blotting, RT-PCR and PCR; “biochemical” assays, such as detecting the presence or absence of a particular peptide, e.g., by immunological means (ELISAs and Western blots). To detect and quantitate RNA, RT-PCR may be employed. Further information about the RNA product may be obtained by Northern blotting. Expression may also be confirmed by specifically identifying the peptide encoded by the transgene or evaluating the phenotypic changes brought about by the expression of the transgene.

In another embodiment, the cells are transfected or transduced in vivo. In one aspect, cells from a mammalian (e.g. human) subject are transduced or transfected in vivo with a vector containing a heterologous nucleic acid for expressing a therapeutic agent thereby delivering the therapeutic agent in situ.

Cells that may be transformed, in vitro or in vivo, include a cell of any tissue or organ type, of any origin (e.g., mesoderm, ectoderm or endoderm). Non-limiting examples of cells include central or peripheral nervous system, such as brain (e.g., neural, glial or ependymal cells) or spine, liver (e.g., hepatocytes, sinusoidal endothelial cells), pancreas (e.g., beta islet cells), lung, kidney, eye (e.g., retinal, cell components), spleen, skin, thymus, testes, lung, diaphragm, heart (cardiac), muscle or psoas, gut (e.g., endocrine), adipose tissue (white, brown or beige), muscle (e.g., fibroblasts), synoviocytes, chondrocytes, osteoclasts, epithelial cells, endothelial cells, salivary gland cells, inner ear nervous cells or hematopoietic (e.g., blood or lymph) cells. Additional examples include stem cells, such as pluripotent or multipotent progenitor cells that develop or differentiate into central or peripheral nervous system, such as brain (e.g., neural, glial or ependymal cells) or spine, liver (e.g., hepatocytes, sinusoidal endothelial cells), pancreas (e.g., beta islet cells), lung, kidney, eye (retinal, cell components), spleen, skin, thymus, testes, lung, diaphragm, heart (cardiac), muscle or psoas, or gut (e.g., endocrine), adipose tissue (white, brown or beige), muscle (e.g., fibroblasts), synoviocytes, chondrocytes, osteoclasts, epithelial cells, endothelial cells, salivary gland cells, inner ear nervous cells or hematopoietic (e.g., blood or lymph) cells.

In accordance with the invention, nucleic acid sequences encoding a dCas9 protein, guide polynucleotides such as crRNA and tracrRNA polynucleotides or sgRNA polynucleotides, and expression cassettes, recombinant lenti-viral vectors, or recombinant adeno-associated virus (rAAV) vectors comprising such nucleic acid sequences can can used to modulate, typically, reduce, inhibit, suppress or decrease expression of a genetic locus of interest. Such molecules include those able to inhibit expression of a target gene involved in treatment of a disease process, thereby reducing, inhibiting or alleviating one or more symptoms of a disease.

In certain embodiments, a method of inhibiting expression of a target protein in a subject includes introducing a first nucleic acid encoding a nuclease defective Cas9 (dCas9) polypeptide and a second nucleic acid encoding a guide polynucleotide (e.g., by way of an rAAV) that encodes or is translated into a dCas9 protein and transcribed into a sgRNA, respectively, in an amount sufficient to reduce, inhibit, suppress or decrease expression of the target protein when administered to a subject. In certain embodiments, an expression cassette comprises a first nucleic acid encoding a dCas9 and a second nucleic acid encoding a guide polynucleotide.

In certain embodiments, a vector, such as a recombinant lenti-vector or a recombinant adeno-associated (rAAV) vector, comprises a first expression cassette comprising a first nucleic acid encoding a nuclease defective Cas9 (dCas9) polypeptide and a second expression cassette comprising a second nucleic acid encoding a guide polynucleotide. The first expression cassette expresses the dCas9 polypeptide and the second expression cassette expresses the guide polynucleotide (e.g., sgRNA). The first or second expression cassette can be on the same vector or on two vectors, e.g., a single AAV or two AAV vectors.

The terms “silencing” or “silenced” used interchangeably herein, means that a targeted gene locus, such as a mutant HTT locus, is inhibited by the presence of the introduced nucleic acid sequence encoding the guide polynucleotide and the nucleic acid encoding the dCas9 polypeptide, such that expression of the targeted gene locus, such as a mutant HTT locus, is reduced.

Reduction of expression can be about 10% to 100% as compared to the level of expression seen when such nucleic acids are not present. Generally, when a gene is silenced, it will have at least 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, 98%, 99% or even 100% reduction in expression as compared to expression when such nucleic acids are not present.

In certain embodiments, a mutant allele, such as a mutant HTT allele, is silenced by the presence of the introduced nucleic acid sequence encoding the guide polynucleotide and the nucleic acid encoding the dCas9 polypeptide. In other embodiments, a mutant HTT allele that comprises a single nucleotide polymorphism (SNP) is silenced by the presence of the introduced nucleic acid sequence encoding the guide polynucleotide and the nucleic acid encoding the dCas9 polypeptide.

Guide polynucleotides, such as crRNAs, tracrRNAs, and sgRNAs, can be produced based upon nucleic acids encoding target gene sequences (e.g., Huntingtin, or HTT), such as nucleic acid encoding mammalian or human HTT. In certain embodiments, guide polynucleotides, such as crRNAs, tracrRNAs, and sgRNAs, can be produced based upon nucleic acids encoding polymorphic forms of target gene sequences, such as single-nucleotide polymorphisms (SNPs) of target genes (e.g., Huntingtin, or HTT), that cause disease (such as Huntington's Disease) when expressed in a subject. Thus, guide polynucleotides, such as crRNAs, tracrRNAs, and sgRNAs, and/or expression cassettes, lenti-vectors, and AAV vectors comprising them can be employed to target a mutant huntingtin allele, such as a huntingtin allele that comprises a single-nucleotide polymorphism, to reduce, inhibit, suppress or decrease, expression of the mutant huntingtin allele.

In some embodiments, the length of the guide polynucleotide less than 70 nucleotides in length. In some embodiments, the guide polynucleotide is at least 10 nucleotides in length. In some embodiments, the length of the guide polynucleotide is between 10 and 70 nucleotides in length. In some embodiments, the length of the guide polynucleotide is between 10 and 60 nucleotides in length. In some embodiments, the length of the guide polynucleotide is between 10 and 50 nucleotides in length. In some embodiments, the length of the guide polynucleotide is between 10 and 40 nucleotides in length. In some embodiments, the length of the guide polynucleotide is between 10 and 30 nucleotides in length. In some embodiments, the guide polynucleotide is 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11 or 10 nucleotides in length.

Nucleic acid sequences encoding guide polynucleotides and the nucleic acid sequences encoding a dCas9 polypeptide can be encoded by a heterologous nucleic acid sequence, and the heterologous nucleic acid sequence can additional elements as set forth herein, such as an expression control element and/or a polyadenylation signal.

Nucleic acid sequences encoding guide polynucleotides and the nucleic acid sequences encoding a dCas9 polypeptide can also be synthesized in vitro, chemically synthesized, or expressed by cells, such as primary cells or immortalized cell lines, that have been transduced or transfected with the nucleic acid sequences.

Particular non-limiting examples of genes (e.g., genomic DNA) that may be targeted with CRISPRi nucleic acid sequences of the invention include, but are not limited to genes associated with polynucleotide repeat diseases such as huntingtin (HTT) gene.

In certain embodiments, genes associated with polynucleotide repeat diseases such as huntingtin (HTT) gene may be targeted with CRISPRi nucleic acid sequences in accordance with the invention. In certain embodiments, polymorphic (e.g. SNP) mutant HTT alleles may be targeted with CRISPRi nucleic acid sequences in accordance with the invention.

Methods and uses of the invention provide a means for delivering (transducing) nucleic acid sequences as set forth herein heterologous nucleic acid sequences (transgenes) into a broad range of host cells, including dividing and non-dividing cells. The recombinant vector (e.g., AAV), vector genomes, methods, uses and pharmaceutical formulations of the invention are additionally useful in a method of administering or delivering a nucleic acid encoding a guide polynucleotide and a dCas9 polypeptide to a subject in need thereof, as a method of treatment. In this manner, the guide polynucleotide and the dCas9 polypeptide may be produced in vivo in a subject.

In general, invention recombinant vector (e.g., AAV), vector genomes, methods and uses may be used to deliver any heterologous nucleic acid (transgene) with a biological effect to treat or ameliorate one or more symptoms associated with any disorder related to undesirable gene expression. Accordingly, invention compositions (e.g., heterologous nucleic acid sequences), methods and uses permit the treatment of genetic diseases, such as Huntington's disease.

In accordance with the invention, in vivo administration and treatment methods and uses are provided that include invention recombinant vector (e.g., AAV), vector genomes, and recombinant virus particles including vector genomes. Methods and uses of the invention are also broadly applicable to diseases amenable to treatment by reducing or decreasing gene expression or function, e.g., gene silencing, knockout or reduction of gene expression (gene knockdown).

Invention methods also include delivering a nucleic acid to CNS (e.g brain) cells, such as HTT-expressing CNS (e.g., brain) cells, by administering an AAV vector with a heterologous nucleic acid inserted between a pair of AAV inverted terminal repeats to such cells. Target cells include neurons in the striatum or cortex in a subject. In one embodiment, a method includes administering to the subject an AAV particle comprising heterologous nucleic acid inserted between a pair of AAV inverted terminal repeats, thereby delivering the nucleic acid to the neuron or other CNS (e.g., brain) cell in the subject.

According to a method of the invention, expression of RNA encoding a polymorphic mutant HTT allele, such as a mutant huntingtin allele that contains a single nucleotide polymorphism near the transcriptional start site of the mutant HTT allele (e.g. within about 500 polynucleotides), can be inhibited, suppressed, or silenced by using the nucleic acids of the invention. For example, production or accumulation of RNA encoding a mutant huntingtin allele can be inhibited, suppressed, or silenced in a cell.

Methods of delivery of viral vectors include injecting the AAV into the subject. Generally, rAAV virions may be introduced into cells of the CNS using either in vivo or in vitro transduction techniques. Suitable methods for the delivery and introduction of transduced cells into a subject are disclosed herein and known to the skilled artisan.

Suitable subjects include mammals, such as humans, as well as non-human mammals. The term “subject” refers to an animal, typically a mammal, such as humans, non-human primates (apes, gibbons, gorillas, chimpanzees, orangutans, macaques), a domestic animal (dogs and cats), a farm animal (poultry such as chickens and ducks, horses, cows, goats, sheep, pigs), and experimental animals (mouse, rat, rabbit, guinea pig). Human subjects include fetal, neonatal, infant, juvenile and adult subjects. Subjects include animal disease models, such as dog and non-human primate models of CNS diseases.

“Treating” as used herein refers to ameliorating at least one symptom of, curing and/or preventing the development of a disease or a condition, such as after development of a pathology or symptom of a disease, such as CNS disease. The term “ameliorate” means a detectable or measurable improvement in a subject's disease, pathology or symptom thereof, or an underlying cellular response. A detectable or measurable improvement includes a subjective or objective decrease, reduction, inhibition, suppression, limit or control in the occurrence, frequency, severity, progression, or duration of the disease, pathology or complication caused by or associated with the disease, or an improvement in a symptom or an underlying cause or a consequence of the disease (pathology), or a reversal of the disease. Treating can include stabilizing the disease, pathology or symptom thereof, or preventing progression, worsening or halting a disease, pathology or symptom, as well as reversing severity of a disease, pathology or symptom or providing an improvement in the disease, pathology or symptom.

Treating Huntington's disease in accordance with the invention includes, for example, an alleviation of signs and/or symptoms of the disease in patients with ongoing disease signs and/or symptoms. Such disease signs or symptoms include, for example, movement disorders such as involuntary or uncontrolled movement of arms, legs, head, face, and/or upper body, jerking or writhing movements (i.e., chorea), involuntary and sustained contracture of muscles (i.e., dystonia), muscle rigidity, slow and uncoordinated fine movements, slow or abnormal eye movements, impaired gait, impaired posture, impaired balance difficulty with the physical production of speech, and difficulty swallowing. Such disease signs or symptoms also include, for example, cognitive, mental, and mood disorders, such as cognitive decline, decline or loss of thinking and/or reasoning skills, decline or loss of memory, concentration, judgment and/or ability to plan and organize, alterations in mood, depression, anxiety, and/or uncharacteristic anger or irritability, and/or obsessive-compulsive behavior.

Treating Huntington's disease in accordance with the invention also includes treating stable and progressive symptoms of Huntington's disease as well as preemptively administering CRISPRi as described herein to delay the onset of one or more symptoms of Huntington's disease. Treating Huntington's disease also includes precluding the appearance of signs and/or symptoms anytime as well as reducing existing signs and/or symptoms and reducing or eliminating existing signs and/or symptoms.

Vectors of the invention can be administered to provide a reduction in at least one symptom associated with Huntington's disease. Accordingly, pharmaceutical compositions of the invention include compositions in which a therapeutic agent (e.g., rAAV) is in an amount effective to achieve the intended therapeutic purpose.

An “effective amount” or “sufficient amount” refers to an amount that provides, in single or multiple doses, alone or in combination, with one or more other compositions (therapeutic agents such as a drug), treatments, protocols, or therapeutic regimens agents, a detectable response of any duration of time (long or short term), an expected or desired outcome in or a benefit to a subject of any measurable or detectable degree or for any duration of time (e.g., for minutes, hours, days, months, years, or cured). The doses of an “effective amount” or “sufficient amount” for treatment (e.g., to ameliorate or to provide a therapeutic benefit or improvement) typically are effective to provide a response to one, multiple or all adverse symptoms, consequences or complications of the disease, one or more adverse symptoms, disorders, illnesses, pathologies, or complications, for example, caused by or associated with the disease, to a measurable extent, although decreasing, reducing, inhibiting, suppressing, limiting or controlling progression or worsening of the disease is a satisfactory outcome.

For a CNS disease, an effective amount would be an amount that improves motor or cognitive function, or reduces, decreases, suppresses or inhibits motor or cognitive impairment or further impairment, deterioration or worsening in motor or cognitive function. In certain embodiments, an effective amount would be an amount that improves wide-based gait, difficulties with balance, speech, swallowing, coordination and/or spasticity; reduces, decreases, suppresses or inhibits wide-based gait, difficulties with balance, speech, swallowing, coordination and/or spasticity; or further impairment, deterioration or worsening of wide-based gait, difficulties with balance, speech, swallowing, coordination and/or spasticity. In certain embodiments, an effective amount would be an amount that improves extracerebellar function, for example, reduces, decreases, suppresses or inhibits deep tendon reflexes and oculomotor abnormalities; or reduces, decreases, suppresses or inhibits further impairment, deterioration or worsening of extracerebellar function, for example, deep tendon reflexes and oculomotor abnormalities.

CNS disease status, progression, etc. can be reflected by the foregoing criteria as well as cerebellar pathology, such as thinning in cerebellas lobules, and cell morphology such as Purkinje cell dendrite retraction. CNS disease status, progression, etc. also can be reflected by prevalence/distribution of mutant HTT alleles and prevalence/distribution other molecular biomarkers, etc., as set forth herein. A reduction, decrease, inhibition or suppression, stabilization or preventing worsening, or normalization or a reversal of any of the foregoing, and the other criteria set forth herein for treating and improvement, as well as the specific criteria set forth in the examples herein, can be indicative of an effective amount (e.g., dose).

Formulations containing vector (e.g., rAAV) particles can contain an effective amount of the rAAV, the effective amount determined by one skilled in the art. The rAAV particles may typically range from about 1% to about 95% (w/w) of the composition, or even higher or lower if appropriate.

An effective amount or a sufficient amount (e.g., dose) can, but need not be, provided in a single administration, may require multiple administrations, and, can but need not be, administered alone or in combination with another composition (e.g., agent), treatment, protocol or therapeutic regimen. For example, the amount may be proportionally increased as indicated by the need of the subject, type, status and severity of the disease treated or side effects (if any) of treatment. In addition, an effective amount or a sufficient amount need not be effective or sufficient if given in single or multiple doses without a second composition (e.g., another drug or agent), treatment, protocol or therapeutic regimen, since additional doses, amounts or duration above and beyond such doses, or additional compositions (e.g., drugs or agents), treatments, protocols or therapeutic regimens may be included in order to be considered effective or sufficient in a given subject. Amounts considered effective also include amounts that result in a reduction of the use of another treatment, therapeutic regimen or protocol.

Accordingly, methods and uses include, among other things, methods and uses that result in a reduced need or use of another compound, agent, drug, therapeutic regimen, treatment protocol, process, or remedy. Thus, in accordance with the invention, methods and uses of reducing need or use of another treatment or therapy are provided.

An effective amount or a sufficient amount need not be effective in each and every subject treated, nor a majority of treated subjects in a given group or population. As is typical for such methods, some subjects will exhibit a greater response, or less or no response to a given treatment method or use.

The amount administered will vary depending on various factors. Doses can vary and depend upon the type of disease, onset, progression, severity, frequency, duration, or probability of the disease to which treatment is directed, the clinical endpoint desired, previous or simultaneous treatments, the general health, physical condition, age, gender, race or immunological competency of the subject and other factors that will be appreciated by the skilled artisan. The dose amount, number, frequency or duration may be proportionally increased or reduced, as indicated by any adverse side effects, complications or other risk factors of the treatment or therapy and the status of the subject. The factors that may influence the dosage and timing required to provide an amount effective or sufficient for providing a therapeutic or prophylactic benefit can be readily determined by the skilled artisan.

The dose to achieve a therapeutic effect, e.g., the dose in vector genomes/per kilogram of body weight (vg/kg), will vary based on several factors including, but not limited to: route of administration, the level of heterologous nucleic acid sequence expression required to achieve a therapeutic effect, the specific disease treated, any host immune response to the viral vector, a host immune response to the heterologous nucleic acid sequence or expression product (protein), and the stability of the protein expressed. One skilled in the art can determine rAAV/vector genome dose range to treat a patient having a particular disease or disorder based on the aforementioned factors, as well as other factors.

Methods of determining effective doses of administration are known to those of skill in the art and will vary with the viral vector, the composition of the therapy, the target cells, and the subject being treated. For example, an effective amount of AAV vector to be administered can be based upon non-human primate or other mammalian studies, or empirically determined.

Single and multiple administrations can be carried out with the dose level and pattern being selected by the treating physician. Both local and systemic administration is contemplated. Administration may be continuous or intermittent. Administration can be effected in one dose, continuously or intermittently throughout the course of treatment.

Administration or in vivo delivery to a subject can be performed prior to or after development of an adverse symptom, condition, complication, etc. caused by or associated with the disease, such as a pathology or symptom of a CNS disease. For example, a screen (e.g., genetic) can be used to identify such subjects as candidates for invention compositions, methods and uses. A screen such as mere observation can also be used to identify subjects having an adverse symptom, condition, complication, etc. caused by or associated with the disease, such as a pathology or symptom of a CNS disease, as subjects appropriate for invention compositions, methods and uses. Such subjects therefore include those screened positive for an insufficient amount or a deficiency in a functional gene product or production of a harmful, deleterious, aberrant, partially functional or non-functional gene product.

Administration or in vivo delivery to a subject in accordance with the methods and uses of the invention as disclosed herein can be practiced within 1-2, 2-4, 4-12, 12-24 or 24-72 hours after a subject has been identified as having the disease targeted for treatment, has one or more symptoms of the disease, or has been screened and is identified as positive as set forth herein even though the subject does not have one or more symptoms of the disease. Of course, methods and uses of the invention can be practiced 1-7, 7-14, 14-21, 21-48 or more days, months or years after a subject has been identified as having the disease targeted for treatment, has one or more symptoms of the disease, or has been screened and is identified as positive as set forth herein.

In certain embodiments, rAAV is administered at a dose of about 0.3-2 ml of 1×10⁵-1×10⁶ vg/ml. In certain embodiments, the rAAV is administered at a dose of about 1-3 ml of 1×10⁷-1×10¹⁴ vg/ml. In certain embodiments, the rAAV is administered at a dose of about 1-2 ml of 1×10⁸-1×10¹³ vg/ml.

In certain embodiments, a dose of rAAV is at least 1×109 vector genomes (vg) per kilogram (vg/kg) of the weight of the subject, at least 1×100 vector genomes (vg) per kilogram (vg/kg) of the weight of the subject, between about 1×10¹⁰ to 1×10¹¹ vg/kg of the weight of the subject, between about 1×10¹¹ to 1×11¹² vg/kg of the weight of the subject, or between about 1×10¹² to 1×10¹³ vg/kg of the weight of the subject.

The vectors can be included in pharmaceutical compositions. Such compositions can optionally include sufficient vector to produce an effective amount of the heterologous nucleic acid, i.e., an amount sufficient to reduce or ameliorate symptoms of a disease state in question or an amount sufficient to confer the desired benefit.

Pharmaceutical compositions include solvents (aqueous such as saline, water, artificial CSF, or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Supplementary active compounds (e.g., surfactants, preservatives, antibacterial, antiviral and antifungal agents) can also be incorporated into the compositions.

The pharmaceutical compositions typically will contain a pharmaceutically acceptable excipient. Such excipients include any pharmaceutical agent that does not itself induce the production of antibodies harmful to the individual receiving the composition, and which may be administered without undue toxicity. Pharmaceutically acceptable excipients include, but are not limited to, sorbitol, Tween80, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles.

To prepare a formulation, the puriied composition can be manufactured, prepared and/or isolated. The composition may then be adjusted to an appropriate concentration.

Pharmaceutical compositions can be formulated to be compatible with a particular route of administration or delivery, as set forth herein or known to one of skill in the art. Thus, pharmaceutical compositions include carriers, diluents, or excipients suitable for administration by various routes.

Pharmaceutical compositions can be administered by a variety of routes including parenteral, including by intravenous and intramuscular routes, as well as by direct injection into the diseased tissue. For example, therapeutic agent (e.g., rAAV) may be directly injected into the brain. Alternatively the therapeutic agent (e.g., rAAV) may be introduced intrathecally for brain and spinal cord conditions. In another example, the therapeutic agent (e.g., rAAV) may be introduced intramuscularly for vectors that traffic to affected neurons from muscle, such as AAV, lentivirus and adenovirus.

Compositions suitable for parenteral administration comprise aqueous and non-aqueous solutions, suspensions or emulsions of the active compound, which preparations are typically sterile and can be isotonic with the blood of the intended recipient. Non-limiting illustrative examples include water, buffered saline, Hanks' solution, Ringer's solution, dextrose, fructose, ethanol, animal, vegetable or synthetic oils. Aqueous injection suspensions may contain substances which increase the viscosity of the suspension, such as sodium carboxymethyl cellulose, sorbitol, or dextran.

Co-solvents and adjuvants may be added to the formulation. Non-limiting examples of co-solvents contain hydroxyl groups or other polar groups, for example, alcohols, such as isopropyl alcohol; glycols, such as propylene glycol, polyethyleneglycol, polypropylene glycol, glycol ether, glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

After pharmaceutical compositions have been prepared, they may be placed in an appropriate container and labeled for treatment. For administration of vectors (e.g., rAAV) as set forth herein, such labeling can include amount, frequency, and method of administration.

Pharmaceutical compositions and delivery systems appropriate for the compositions, methods and uses of the invention are known in the art (see, e.g., Remington: The Science and Practice of Pharmacy (2003) 20th ed., Mack Publishing Co., Easton, Pa.; Remington's Pharmaceutical Sciences (1990) 18th ed., Mack Publishing Co., Easton, Pa.; The Merck Index (1996) 12th ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 1th ed., Lippincott Williams & Wilkins, Baltimore, Md.; and Poznansky et al., Drug Delivery Systems (1980), R. L. Juliano, ed., Oxford, N.Y., pp. 253-315).

The compositions may be conveniently prepared or provided in discrete unit dosage forms and may be prepared by any of methods well known to pharmacy. Such methods may include the step of bringing into association the vector (therapeutic agent) with liquid carriers, solid matrices, semi-solid carriers, finely divided solid carriers or combinations thereof, and then, if necessary, introducing or shaping the product into the desired delivery system.

A “unit dosage form” as used herein refers to physically discrete units suited as unitary dosages for the subject to be treated; each unit containing a predetermined quantity optionally in association with a pharmaceutical carrier (excipient, diluent, vehicle or filling agent) which, when administered in one or more doses, is calculated to produce a desired effect (e.g., prophylactic or therapeutic effect). Unit dosage forms may be within, for example, ampules and vials, which may include a liquid composition, or a composition in a freeze-dried or lyophilized state; a sterile liquid carrier, for example, can be added prior to administration or delivery in vivo. Individual unit dosage forms can be included in multi-dose kits or containers. Recombinant vector (e.g., rAAV), recombinant virus particles, and pharmaceutical compositions thereof can be packaged in single or multiple unit dosage form for ease of administration and uniformity of dosage.

The invention provides kits with packaging material and one or more components therein. A kit typically includes a label or packaging insert including a description of the components or instructions for use in vitro, in vivo, or ex vivo, of the components therein. A kit can contain a collection of such components, e.g., a filler or stuffer alone, or in combination or be a component of, a heterologous nucleic acid sequence, recombinant vector, virus (e.g., AAV) vector, and optionally in combination with a second active, such as another compound, agent, drug or composition.

A kit refers to a physical structure housing one or more components of the kit. Packaging material can maintain the components sterilely, and can be made of material commonly used for such purposes (e.g., paper, corrugated fiber, glass, plastic, foil, ampules, vials, tubes, etc.).

Labels or inserts can include identifying information of one or more components therein, dose amounts, clinical pharmacology of the active ingredient(s) including mechanism of action, pharmacokinetics and pharmacodynamics. Labels or inserts can include information identifying manufacturer, lot numbers, manufacture location and date, expiration dates. Labels or inserts can include information identifying manufacturer information, lot numbers, manufacturer location and date. Labels or inserts can include information on a disease for which a kit component may be used. Labels or inserts can include instructions for the clinician or subject for using one or more of the kit components in a method, use, or treatment protocol or therapeutic regimen. Instructions can include dosage amounts, frequency or duration, and instructions for practicing any of the methods, uses, treatment protocols or prophylactic or therapeutic regimes described herein.

Labels or inserts can include information on any benefit that a component may provide, such as a prophylactic or therapeutic benefit. Labels or inserts can include information on potential adverse side effects, complications or reactions, such as warnings to the subject or clinician regarding situations where it would not be appropriate to use a particular composition. Adverse side effects or complications could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities.

Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a bar-coded printed label, a disk, optical disk such as CD- or DVD-ROM/RAM, DVD, MP3, magnetic tape, or an electrical storage media such as RAM and ROM or hybrids of these such as magnetic/optical storage media, FLASH media or memory type cards.

The term “about” at used herein refers to a values that is within 10% (plus or minus) of a reference value.

The terms “a” and “an” and “the” and similar referents in the context of describing the invention are to be construed to cover both the singular and the plural, unless otherwise indicated herein or clearly contradicted by context. Thus, for example, reference to “a vector” includes a plurality of such vectors, reference to “a virus” or “particle” includes a plurality of such virions/particles and reference to “AAV or rAAV particle” includes a plurality of such AAV or rAAV particles.

The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

All applications, publications, patents and other references, GenBank citations and ATCC citations cited herein are incorporated by reference in their entirety. In case of conflict, the specification, including definitions, will control.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All of the features disclosed herein may be combined in any combination. Each feature disclosed in the specification may be replaced by an alternative feature serving a same, equivalent, or similar purpose. Thus, unless expressly stated otherwise, disclosed features are an example of a genus of equivalent or similar features.

All numerical values or numerical ranges include integers within such ranges and fractions of the values or the integers within ranges unless the context clearly indicates otherwise. Thus, to illustrate, reference to 80% or more identity, includes 81%, 82%, 83%, 84%, 85%, 86%, 87%, 88%, 89%, 90%, 91%, 92%, 93%, 94%, etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000, includes ranges of 10-50, 50-100, 100-1,000, 1,000-3,000, 2,000-4,000, etc.

The invention is generally disclosed herein using affirmative language to describe the numerous embodiments and aspects. The invention also specifically includes embodiments in which particular subject matter is excluded, in full or in part, such as substances or materials, method steps and conditions, protocols, or procedures. For example, in certain embodiments or aspects of the invention, materials and/or method steps are excluded. Thus, even though the invention is generally not expressed herein in terms of what the invention does not include aspects that are not expressly excluded in the invention are nevertheless disclosed herein.

Embodiments of the invention are described herein. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the elements in all possible variations thereof is encompassed by the invention unless otherwise indicated herein or otherwise clearly contradicted by context. Accordingly, the following examples are intended to illustrate but not limit the scope of the invention claimed.

### EXAMPLES

### Example 1

The guide sgRNA sequences are composed of two elements (complementary sequence+scaffold sequence) The guide sequences in example 2 below are the complementary sequences that bind to the genomic DNA. These sequences bind nearby to an adjacent protospacer motif (PAM). This PAM is a TGG sequence in our case (Canonical sequence is NGG), and is crucial to activate Cas9 and induce dsDNA breaks.

In the case of the mutant HTT allele, a single nucleotide motif (SNP) is reported on the second G. So, depending on the nucleotide variation the guide can bind to the sequence or not. If the allele as a G, the guide binds, if is a different nucleotide it does not bind. This provides for allele specific targeting.

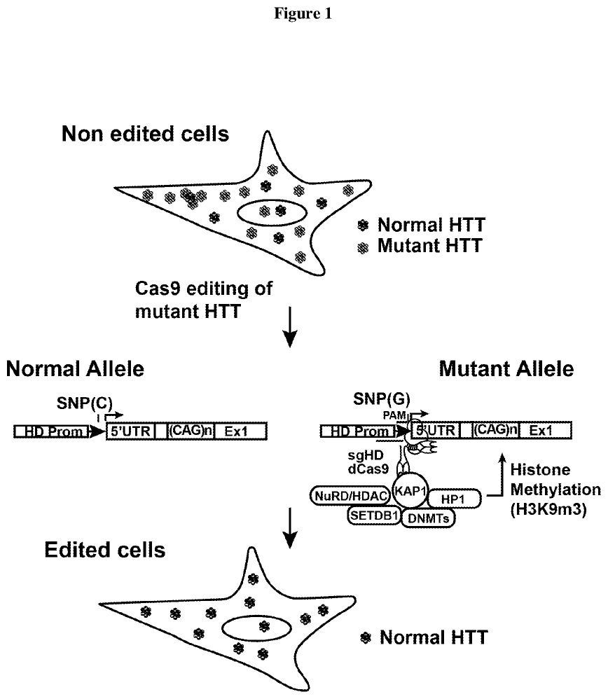

This complementary sequence is part of a longer RNA sequence that contains a specific sequence (Scaffold sequence) to bind to a Cas9 protein. The sequence that binds to Cas9 fold in two RNA loops that have been modified to contain a MS2 sequences. These MS2 sequences are binding sites for a chimeric protein that contain a domain called MCP that recognize and bind the MS2 sequence fused to a KRAB domain sequence from KOX1 protein (see, FIG. 1). The KRAB domain recruits KAP1. That acts as a scaffold to bind several proteins involved in chromatin remodeling (the other proteins illustrated in FIG. 1). Overall, a complex is made that induces histone 3 methylation (H3K9m3), and repression of transcription at the mutant HTT locus. Because the polynucleotide recognizes only the sites that have the PAM motif, when the SNP is present in heterozygosity only one HTT allele, the mutant allele, is repressed and the other HTT allele that does not have the PAM motif is not.

### Example 2

Several SNP-dependent PAM motifs were identified upstream and downstream of HTT exon1. A single allele of HTT exon can be effectively eliminated or silenced when using sgRNA/Cas9 complexes that recognize SNP-dependent PAM motifs that are present in heterozygosity.

The HTT genomic targeted sequence, GCGCAGCGTCTGGGACGCAAGGCGCCG, is located in the 5′ untranslated region (UTR, see Table 1). TGG is the PAM motif. The guide polynucleotides that reduce expression of mutant HTT protein, except for seq 3nt27, are as follows: GACGCAAGGCGCCG (TGG) Guide 3nt14; GTCTGGGACGCAAGGCGCCG(TGG)3nt20; GCGTCTGGGACGCAAGGCGCCG (TGG) 3nt22; GCAGCGTCTGGGACGCAAGGCGCCG (TGG) 3nt2; and GCGCAGCGTCTGGGACGCAAGGCGCCG (TGG) 3nt27.

TABLE 1
Representative SNPs upstream of human HTT exon - 1

| | | 1000 genome | | |
|---|---|---|---|---|
| | | data | | |
| | SNP | Ref. | Min. | MAF > |
| Location | Sequence Variation | Allele | Allele | 0.05 |
| Promoter | GTCTGCGTCAGGGTTTCCTTCTTTT[C/G] | C | G | 0.1074 |
| | CAGCCCCACCCCGCGTGCATCCCAC | | | |
| Promoter | TCCCTCATTCAGGTTGATGTCCTAA[C/G] | G | C | 0.1372 |
| | CCCCAGAACCTCAGAATGGGATTGT | | | |
| Promoter | TGTGGCCTGGCTAAAGTAGGCTTTA[C/G] | C | G | 0.1575 |
| | TGGGCTCCTCTCTGCCTGCATCACC | | | |
| Promoter | GGGCGCAGGCCCATGCGGAAAGGAT[A/C/G] | G | C/T | 0.226 |
| | CCCCGCCGACGCCTGGAGCGGGGCG | | (Rev | |
| | | | Strand) | |
| Promoter | attacagtctcaccacgccccgtcc[C/G] | C | G | 0.1082 |
| | CTCTCCGTTGAGCCCCGCGCCTTCG | | | |
| 5′UTR | AGCGTCTGGGACGCAAGGCGCCGTG[A/G] | G | A | 0.1581 |
| | GGGCTGCCGGGACGGGTCCAAGATG | | | |

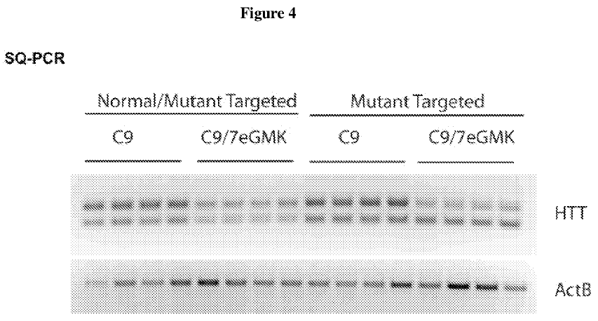

Viral delivery of SNP-dependent Cas9/sgRNA complexes in the striatum of BACHD mice effectively selectively targets mutant HTT in an allele specific manner to reduce levels of the mutant HTT (FIG. 4).

What is claimed:

1. An expression cassette comprising a first nucleic acid encoding a nuclease defective Cas 9 (dCas9) polypeptide; and a second nucleic acid encoding a guide polynucleotide that targets the dCas 9 polypeptide to a sequence positioned 5′ of the transcriptional start site of an allele encoding mutant HTT protein.

2. The expression cassette of claim 1, wherein the allele encoding mutant HTT protein is heterozygous with respect to an allele encoding normal/wild-type HTT protein.

3. The expression cassette of claim 1 or 2, wherein the guide polynucleotide is specific for a single nucleotide polymorphism (SNP) present in the allele encoding mutant HTT protein.

4. The expression cassette of any of claims 1 to 3, wherein the dCas 9 polypeptide is fused to a transcription repressor domain.

5. The expression cassette of claim 4, wherein the transcription repressor domain comprises a Krüppel associated box (KRAB), a Mad mSIN3 interaction domain (SID) or an ERF repressor domain (ERD).

6. The expression cassette of any of claims 1 to 5, wherein the sequence positioned 5′ comprises a 5′ untranslated region or a transcriptional regulatory region of the allele encoding mutant HTT protein comprises a promoter or enhancer.

7. The expression cassette of any of claims 1 to 6, wherein the guide polynucleotide binds to a sequence within 200 nucleotides of the transcriptional start site of the allele encoding mutant HTT protein.

8. The expression cassette of any of claims 1 to 7, wherein the guide polynucleotide is specific for a single nucleotide polymorphism (SNP) present in the sequence positioned 5′ of the allele encoding mutant HTT protein.

9. The expression cassette of claim 8, wherein the single nucleotide polymorphism (SNP) present in the sequence positioned 5′ of the allele encoding mutant HTT protein is a protospacer adjacent motif (PAM).

10. The expression cassette of any of claims 1 to 9, wherein the sequence positioned 5′ is within about 0-100 nucleotides from a protospacer adjacent motif (PAM) sequence.

11. The expression cassette of any of claims 1 to 10, wherein the guide polynucleotide is 10 to 50 nucleotides in length and binds to at least 10 nucleotides of the sequence set forth as: GCGCAGCGTCTGGGACGCAAGGCGCCG.

12. The expression cassette of any of claims 1 to 11, wherein the guide polynucleotide is 10 to 30 nucleotides in length and binds to the sequence set forth as: GCGCAGCGTCTGGGACGCAAGGCGCCG.

13. The expression cassette of any of claims 1 to 12, wherein the second nucleic acid comprises any of the following sequences: GACGCAAGGCGCCG, GTCTGGGACGCAAGGCGCCG, GCGTCTGGGACGCAAGGCGCCG or GCAGCGTCTGGGACGCAAGGCGCCG.

14. The expression cassette of any of claims 1 to 13, wherein the guide polynucleotide is an RNA sequence.

15. The expression cassette of any of claims 1 to 14, wherein the mutant HTT protein has greater than 36 poly-glutamine residue repeats.

16. The expression cassette of any of claims 1 to 14, wherein the mutant HTT protein has between about 36-50 poly-glutamine residue repeats.

17. The expression cassette of any of claims 1 to 14, wherein the mutant HTT protein has greater than 50 poly-glutamine residue repeats.

18. A recombinant viral vector comprising the expression cassette of any of claims 1 to 17.

19. A recombinant lenti-viral vector comprising the expression cassette of any of claims 1 to 17.

20. A recombinant adeno-associated viral (rAAV) vector comprising the expression cassette of any of claims 1 to 17 and one or more AAV inverted terminal repeat (ITR) sequences.

21. The rAAV vector of claim 20, wherein said rAAV vector comprises one or more of:
a) an AAV capsid; and
b) one or more AAV inverted terminal repeats (ITRs), wherein said AAV ITR(s) flanks the 5′ or 3′ terminus of said first nucleic acid and/or said second nucleic acid.

22. The AAV vector of claim 20 or 21, wherein said AAV capsid serotype comprises a modified or variant AAV VP1, VP2 and/or VP3 capsid having 90% or more sequence identity to AAV1, AAV2, AAV3, AAV3B, AAV4, AAV5, AAV6, AAV7, AAV8, AAV9, AAV10, AAV11, Rh10, Rh74, or AAV-2i8 VP1, VP2 and/or VP3 sequences, or a capsid having 95% or more sequence identity to AAV1, AAV2, AAV3, AAV4, AAV5, AAV6, AAV7, AAV8, AAV9, AAV10, AAV11, AAV12, Rh10, Rh74, AAV3B or AAV-2i8 VP1, VP2 and/or VP3 sequences, or a capsid having 100% sequence identity to AAV1, AAV2, AAV3, AAV3B, AAV4, AAV5, AAV6, AAV7, AAV8, AAV9, AAV10, AAV11, Rh10, Rh74, or AAV-2i8 VP1, VP2 and/or VP3 sequences.

23. The AAV vector of any of claim 22, wherein said ITRs comprise one or more ITRs of any of: AAV1, AAV2, AAV3, AAV3B, AAV4, AAV5, AAV6, AAV7, AAV8, AAV9, AAV10, AAV11, Rh10, or Rh74 serotypes, or a combination thereof.

24. A pharmaceutical composition comprising a plurality of AAV vectors of any of claims 20 to 23 in a biologically compatible carrier or excipient.

25. The pharmaceutical composition of claim 24, further comprising empty AAV capsids.

26. The pharmaceutical composition of claim 25, wherein the ratio of said empty AAV capsids to said AAV vector is within or between about 100:1-50:1, from about 50:1-25:1, from about 25:1-10:1, from about 10:1-1:1, from about 1:1-1:10, from about 1:10-1:25, from about 1:25-1:50, or from about 1:50-1:100.

27. The pharmaceutical composition of any of claims 22 to 26, further comprising a surfactant.

28. A method of reducing or inhibiting expression of mutant HTT protein in a subject, comprising administering an amount of nuclease defective Cas9 (dCas9)-guide polynucleotide complex that binds to a genomic sequence comprising a mutant HTT allele to a human in an amount effective to reduce or inhibit expression of mutant HTT protein, thereby reducing or inhibiting expression of the mutant HTT protein in the human.

29. A method of reducing or inhibiting expression of mutant HTT protein, comprising administering an amount of expression cassette, viral vector, lenti-viral vector, AAV vector, or pharmaceutical composition of any of claims 1 to 27 to said human with Huntington's disease, thereby reducing or inhibiting expression of the mutant HTT protein.

30. A method of treating a human for Huntington's disease, comprising administering an amount of expression cassette, viral vector, lenti-viral vector, AAV vector, or pharmaceutical composition of any of claims 1 to 27 to said human with Huntington's disease, effective to reduce or inhibit expression of mutant HTT protein in the human.

31. The method of any of claims 28 to 30, wherein the guide polynucleotide is specific for a single nucleotide polymorphism (SNP) present in the allele encoding mutant HTT protein of said human.

32. The method of any of claims 28 to 30, wherein the guide polynucleotide is specific for a single nucleotide polymorphism (SNP) present in the allele encoding mutant HTT protein that is absent from normal/wild-type HTT allele of said human.

33. The method of any of claims 28 to 32, wherein the human has a gene encoding or expresses a mutant HTT protein having greater than 36 poly-glutamine residue repeats.

34. The method of any of claims 28 to 32, wherein the human has a gene encoding or expresses a mutant HTT protein having between about 36-50 poly-glutamine residue repeats.

35. The method of any of claims 28 to 32, wherein the human has a gene encoding or expresses a mutant HTT protein having greater than 50 poly-glutamine residue repeats.

36. The method of any of claims 28 to 35, wherein said AAV vector is administered to said human intravenously, intraarterially, intra-cavity, intramucosally, or via catheter.

37. The method of any of claims 27 to 36, wherein said AAV vector is administered to said human in a range from about 1×10⁸ to about 1×10¹⁴ vector genomes per kilogram (vg/kg) of the weight of said human.

38. The method of any of claims 28 to 37, wherein said method reduces, decreases, inhibits one or more symptoms of the disease; or prevents or reduces progression or worsening of one or more symptoms of the disease; or stabilizes one or more symptoms of the disease; or improves one or more symptoms of the disease.

39. The method of claim 38, wherein said one or more symptoms of the disease are selected from the group consisting of: uncontrolled movement of arms, legs, head, face and/or upper body; cognitive decline; decline or loss of thinking and/or reasoning skills; decline or loss of memory, concentration, judgment and/or ability to plan and organize; alterations in mood; depression, anxiety, and/or uncharacteristic anger or irritability; and/or obsessive-compulsive behavior.
